(19) United States
(12) Patent Application Publication
(10) Pub. No.: US 2017/0188618 A1
(43) Pub. Date: Jul. 6, 2017

(54) STABLE EMULSIONS FROM DIPEPTIDE DERIVATIVES

(71) Applicant: UNIVERSITY OF STRATHCLYDE, Glasgow (GB)

(72) Inventors: Rein ULIJN, Glasgow (GB); Tell TUTTLE, Glasgow (GB); Ines MOREIRA, Glasgow (GB); Gary SCOTT, Glasgow (GB); Paul John MCKNIGHT, Stonehaven (GB); Martin Edwin RUCK, Stonehaven (GB)

(21) Appl. No.: 15/312,943

(22) PCT Filed: May 22, 2015
(86) PCT No.: PCT/GB2015/051516

§ 371 Date: Nov. 21, 2016

(30) Foreign Application Priority Data
May 22, 2014 (GB) 1409145.8

(51) Int. Cl. A23L 33/175; A61K 9/107; A61K 38/00; A23L 29/10

(52) U.S. Cl. CPC A23L 33/175; A23L 29/10; A61K 9/107; A61K 38/00

(57) ABSTRACT

The present invention relates to the formation of emulsions using small amphipathic molecules, such as peptides which self-assemble to form an interface between at least two substantially immiscible liquids. The emulsions may find application is a variety of technological fields, such as in food, cosmetics, life style products, coating, catalysis, encapsulation, drug delivery and/or cell assays. There is also provided a method of making such emulsions, as well as methods of tailoring the stability of the emulsions for particular applications.

### FIELD OF THE INVENTION

The present invention relates to the formation of emulsions using small amphipathic molecules, such as peptides which self-assemble to form an interface between at least two substantially immiscible liquids. The emulsions may find application is a variety of technological fields, such as in food, cosmetics, life style products, coating, catalysis, encapsulation, drug delivery and/or cell assays. There is also provided a method of making such emulsions, as well as methods of tailoring the stability of the emulsions for particular applications. There is also provided a screening method for identifying peptides which are expected to be capable of self-assembly and may be of use in the formation of emulsions.

### BACKGROUND OF THE INVENTION

Surfactant-based emulsions for encapsulation and phase separation have been extensively utilized in food, cosmetics, coating, catalysis, encapsulation, drug delivery and cell assays. Development of new interfacial stabilization strategies is the key to the advancement of next generation emulsification technologies. Traditional surfactants have disadvantages including toxicity, limited stability towards temperature, pH and salts. A number of approaches, using biocompatible co-polymers, lipids and polypeptides as novel surfactants; bio-macromolecules and proteins that form networked films; or Pickering emulsion based on solid particles and polymersomes have been developed to complement traditional emulsion systems. Nevertheless, there is the need for further emulsion systems.

### SUMMARY OF THE INVENTION

The present invention is based on the use of small amphipathic molecules, such as aromatic group substituted amino acids and peptides to produce interfacial networks to stabilize emulsions. The present invention is also based on a computational screening method which allows the identification of peptides which are expected to be able to self-assemble and hence may be of use in the development of emulsions. Thus, as an alternative to emulsion stabilization by absorption of surfactants, the present invention provides nanostructured networks at interfaces as versatile emulsion stabilizing systems. The approach combines tuneable properties, through molecular design by taking advantage of a balance between intermolecular aromatic π-stacking and hydrogen bond interactions, with long-term high stability at elevated temperature and in the presence of salts, when compared with traditional surfactant sodium dodecyl sulfate (SDS). It is also possible to aggregate or disaggregate the emulsions by enzymatic hydrolysis and other catalytic means to, for example, remove a group or groups and stabilise or destabilise the emulsion, and/or applying of chemical/physical modifications, such as altering pH and/or temperature in order to stabilise or destabilise the emulsion.

Self-assembling small molecules are increasingly investigated as alternatives to polymers as structured materials and gels with advantages such as stimuli-responsiveness biocompatibility, etc. In one embodiment, the present invention is concerned with the, self-assembly of aromatic group substituted amino acids and peptide amphiphiles, containing a hydrophilic short (e.g. di- or tri-) peptide sequence with the N-terminus capped by a hydrophobic synthetic aromatic moiety. In another embodiment, the peptides may simply include aromatic groups naturally present on amino acids which are part of a particular peptide. The inventors show that these molecules are versatile building blocks for the production, via molecular self-assembly, of nanostructures with a variety of morphologies and properties, including localized assembly in microdroplets.

In accordance with a first aspect, the present invention provides an emulsion comprising a self-assembled network of amphipathic amino acids or peptides formed at an interface between at least two substantially immiscible liquids.

As used herein, the term “emulsion” refers to a suspension or dispersion of a first liquid suspended or dispersed in a second liquid in which the first liquid is poorly soluble or non-miscible, that is substantially or fully immiscible with the second liquid. By substantially we mean that at least 90%, 95%, or 98% of the two liquids are immiscible, the first liquid is referred to as the dispersed phase and the second liquid is referred to as the continuous phase. The dispersed phase may form droplets which are dispersed throughout the continuous phase in a heterogeneous or homogeneous manner. Illustrative examples of emulsions include oil-in-water/aqueous solution emulsions in which the oil forms the dispersed phase and the water/aqueous solution forms the continuous phase, and water-in-oil emulsions in which the water forms the dispersed phase and the oil forms the continuous phase. In addition, “multiple emulsions” may be formed in which droplets of a first discontinuous phase contain smaller droplets of a second discontinuous phase, which may or may not be similar in composition to the continuous phase containing the first discontinuous phase. Illustrative examples of multiple emulsions include water-in-oil-in-water emulsions in which the oil forms the first discontinuous phase and water forms the second discontinuous phase, and oil-in-water-in-oil emulsions in which the water forms the first discontinuous phase and oil forms the second discontinuous phase. It may be desirable to trap and hence be able to emulsify other agents, such as drugs, dyes, flavour enhancers, pesticides and the like in the droplets.

The present invention may find particular application in the food industry where emulsions are used to great effect. Such emulsion may include edible oils or fats, in particular vegetable oils or fats. Oils which may be of particular use in food based applications include coconut oil, palm oil, palm kernel oil, olive oil, soybean oil, canola oil (rapeseed oil), pumpkin seed oil, corn oil, sunflower oil, safflower oil, peanut oil, grape seed oil, sesame oil, argan oil, rice bran oil and other vegetable oils, as well as animal-based oils like butter and lard.

The amino acids and peptides of the present invention are amphipathic and have low molecular masses, containing a small number of amino acids. The peptides typically comprise between 2-5 amino acids. The peptides may more preferably be 2-4 amino acids in length. In one embodiment the peptides are dipeptides or tripeptides. The term “amphipathic” refers to peptides or molecules having both hydrophilic and hydrophobic regions. “Amphipathic” and “amphiphilic” are synonymous and are used interchangeably herein. The term “hydrophilic” refers to a molecule or portion of a molecule that is attracted to water and other polar solvents. A hydrophilic molecule or portion of a molecule is polar and/or charged or has an ability to form interactions such as hydrogen bonds with water or polar solvents. The term “hydrophobic” refers to a molecule or portion of a molecule that repels or is repelled by water and other polar solvents. A hydrophobic molecule or portion of a molecule is non-polar, does not bear a charge and is attracted to non-polar solvents.

The peptides of the present invention may be prepared by methods known in the art, such as solid phase synthesis or solution phase synthesis using Fmoc or Boc protected amino acid residues, which may subsequently be removed if appropriate. Alternatively, the peptides may be prepared by recombinant techniques as known in the art using, for example, standard microbial culture technology, genetically engineered microbes and recombinant DNA technology (Sambrook and Russell, Molecular Cloning: A Laboratory Manual (3 <rd>Edition), 2001, CSHL Press). The peptides may also be obtained through enzymic digestion of natural proteins or recombinantly expressed proteins or larger peptide sequences. Furthermore, they may be produced by enzymatic peptide synthesis. The peptides may be further modified following synthetic or recombinant synthesis described above.

As used herein, the term “amino acid” refers to an [alpha]-amino acid or a [beta]-amino acid and may be a L- or D-isomer. The amino acid may be a naturally occurring or non-naturally occurring amino acid. The amino acid may also be further substituted in the [alpha]-position or the [beta]-position with a group, which may be (hetero)-aromatic, aliphatic, may contain hydrogen bond donors or acceptors. These could be fluorescent, (semi-) conducting or bioactive groups such as saccharides, nucleotides, and the like.

Suitable [beta]-amino acids include conformationally constrained [beta]-amino acids. Cyclic [beta]-amino acids are conformationaly constrained and are generally not accessible to enzymatic degradation. Suitable cyclic [beta]-amino acids include, but are not limited to, cis- and trans-2-aminocyclopropyl carboxylic acids, 2-aminocyclobutyl and cyclobutenyl carboxylic acids, 2-aminocyclopentyl and cyclopentenyl carboxylic acids, 2-aminocyclohexyl and cyclohexenyl carboxylic acids and 2-amino-norbornane carboxylic acids and their derivatives.

The term “non-naturally occurring amino acid” as used herein, refers to amino acids having a side chain that does not occur in the naturally occurring (gene encoded) L-[alpha]-amino acids. Examples of non-natural amino acids and derivatives include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, citrulline, sarcosine, 4-amino-3-hydroxy-6-methylheptanoic acid, 2-thienyl alanine and/or D-isomers of amino acids.

In a one embodiment, the amino acids are modified or the peptides may comprise a modified N-terminus. That is, the N-terminal amino acid may comprise a modified group typically bound to the N-terminal amino acid by way of an amide or other suitable bond. Similarly in the case of a single amino acid, the amino acid may be modified by way of an amide or other suitable bond. Preferably the modified group has an overall hydrophobic nature. By this it is understood that the group may have hydrophobic and hydrophilic properties, but overall the group is understood to be hydrophobic in nature. Examples of hydrophobic groups include —CH2- chains and hydrocarbon ring structures.

More preferably, the modifying group may comprise an aromatic group or groups. The aromatic groups may comprise a single or multiple ring structures (e.g. polycyclic aromatic hydrocarbons) and may comprise heterocylic and/or homocyclic ring structures. Typically the aromatic group may comprise one, two, three, four or more fused ring structures, wherein each ring may be identical or different. Representative groups include anthracene, acenaphthene, fluorene, phenalene, tetracene, pyrene, phenanthrene, naphtalene and chysene, phenylacetyl, as well as heterocyclic structures, such as purine, pyrimidine, pteridine, alloxazine, phenoxazine and phenothiazine. The aromatic groups may be bonded to the amino acid through a substituent present on one or more of the aromatic rings. Such a substituent may comprise a reactive C1-C4 alkyl, alkyloxy, alkylamino, phosphate, carboxylic acid, amino, alcohol, N-hydroxysuccinimide, hydroxybenzotriazole, halide, or 1-Hydroxy-7-azabenzotriazole and the like group(s) known in the art. Preferred aromatic groups may be based on furene, pyrene, purine and pyrimidine containing structures, such as fluorenylmethyloxycarbonyl (FMOC), 9-fluorenylmethyl succinimidyl carbonate (FMOC-)Su), C1-C4 alkyl substituted pyrene, and natural and synthetic nucleotides known in the art. Typically total molecular weight of any aromatic group or groups will be less than 500 molecular weight, such as less than 400 molecular weight, or oven 300 molecular weight.

In some embodiments the peptides comprise in addition to the above, or as an alternative, modifications at the N-terminus, C-terminus and/or on the peptide backbone. For example, the N-terminus or C-terminus of a peptide may be modified or a side chain of an amino acid residue within the peptide backbone may be modified. Examples of suitable N-terminus modification include, but are not limited to, acylation with a carboxylic acid containing a straight chain or branched alkyl group or an aryl group. Suitable alkyl groups include, but are not limited to, methyl, ethyl, propyl, isopropyl, butyl, isobutyl, pentyl, hexyl, heptyl, octyl, nonyl, decyl, dodecyl, tetradecyl, hexadecyl and octadecyl. The free amino group at the N-terminus of the peptide may also be modified by addition of other modifying groups known in the art, including, but not limited to, formyl or benzoxycarbonyl groups. Modification of the N-terminus by acylation with a carboxylic acid containing a suitable hydrophobic group may allow enhanced affinity of the peptide for a fluid interface. The free amino group of the peptide may also be modified with additional functional moieties such as metal-binding, fluorescent, electroconducting, semiconducting or spectroscopically or biologically active species, by using suitably activated derivatives of molecules such as aminocoumarin, biotin, fluorescein, diethylenetriaminepentaacetate, hydrazinonicotinamide or 4-methyl-coumaryl-7-amide, thus providing additional functionality to the peptide.

Examples of suitable C-terminus modification include, but are not limited to, amidation with ammonia or an amine containing a straight chain or branched alkyl group or aryl group or esterification with an alcohol containing straight chain or branched alkyl group or with a phenol or aromatic alcohol. Suitable alkyl groups include, but are not limited to, methyl, ethyl, propyl, isopropyl, butyl, isobutyl, pentyl, hexyl, heptyl, octyl, nonyl, decyl, dodecyl, tetradecyl, hexadecyl and octadecyl. Modification of the C-terminus by amidation with an amine containing a suitable hydrophobic group may allow enhanced affinity of the peptide for a fluid-fluid interface. The free carboxylate group at the C-terminus of the peptide may also be modified by addition of other modifying groups known in the art, including but not limited to, N-oxysuccinimide.

Side chain carboxylate groups of amino acid residues within the peptide, for example the side chain carboxylates of aspartate or glutamate residues, may also be modified by amidation with ammonia or an amine containing a straight chain or branched alkyl group or aryl group or by esterification with an alcohol containing a straight chain or branched alkyl group, a phenol or an aromatic alcohol. Suitable alkyl groups include, but are not limited to, methyl, ethyl, propyl, isopropyl, butyl, isobutyl, pentyl, hexyl, heptyl, octyl, nonyl, decyl, dodecyl, tetradecyl, hexadecyl and octadecyl.

Side chain alcohol or phenol groups of amino acid residues within the peptide, for example side chain alcohol or phenol groups of serine, threonine or tyrosine residues, or free amino groups of lysine residues; side chain free amino groups of amino acid residues, such as asparagine, glutamine, lysine and arginine within the peptide; or side chain free thiol groups of amino acid residues within the peptide, including but not limited to side chain thiol groups of cysteine residues, may also be modified by esterification with a carboxylic acid containing a straight chain or branched alkyl group or aryl group. Suitable alkyl groups include, but are not limited to, methyl, ethyl, propyl, isopropyl, butyl, isobutyl, pentyl, hexyl, heptyl, octyl, nonyl, decyl, dodecyl, tetradecyl, hexadecyl and octadecyl. Modification of side chain alcohol or phenol groups of amino acid residues within the peptide by esterification with a carboxylic acid containing a suitable hydrophobic group may allow enhanced affinity of the peptide for a fluid interface. Side chain alcohol or phenol groups of amino acid residues within the peptide may also be reversibly modified by enzymatic or chemical phosphorylation, thus altering the charge on the peptide, as well as the ability of the peptide to bind certain metal ions.

Exemplary peptides for use in accordance with the present invention include the peptides YL, YA, YS, FF, FFF and YL (using the common one-letter code to identify the amino acids), which have been modified at the N-terminal amino acid (i.e Y in the case of YL, for example) to include an FMOC or pyrene group. See scheme 1c for further details.

Through computational screening, the present inventors have identified unmodified peptides, such as tri-peptides, which were expected and later confirmed empirically to self-aggregate and hence be capable of forming emulsions. Thus, in a further embodiment, there is provided an emulsion comprising unmodified peptides, such as a tripeptides, wherein the peptides from a self-assembled network at an interface between at least two substantially immiscible liquids.

The term unmodified is to be understood as referring to peptides formed from natural or unnatural amino acids, which do not include any additional modifications to the chemical structure of the amino acids forming the peptide, either at the N or C terminus of the peptides, or along the backbone of the peptide itself. Exemplary unmodified peptides for use in forming emulsions in accordance with the present invention include the tripeptides KYW, KFF, KYF, FFD and DFF (using the common one-letter code to identify the amino acids).

The present inventors used previously described computational techniques and adapted the techniques for use in relation to the formation of emulsion. The previously described techniques are described in Frederix et al. 2011 and 2015, to which the skilled reader is directed and the entire contents of which are incorporated herein.

In summary, the computational screening protocol, reported previously, in Frederix et al. 2011 and 2015, may be applied to identify peptides that are expected to self-assemble in water. From an initial screen, a subset of peptides may be identified that showed a potential to form fibers and bilayer structures, which may be considered as a pre-requisite for the compounds to act as emulsifiers. This initial screen allows the selection of peptides that were simulated, using the MARTINI coarse-grained force field, Marrink et al 2007, for a further 9.6 μs in both water and immiscible water/solvent solutions, in order to identify peptides which may be expected to form emulsions between at least two substantially immiscible liquids.

Thus in a further aspect, present invention provides a virtual screening method which allows all 8000 tripeptide sequences to be studied in a virtual manner and their propensity for emulsion formation to be estimated such that classes of peptides, such as tripeptides, with certain properties that renders them most likely to form emulsions may be identified.

This aspect of the present invention is based on the development of a method of virtual screening for the propensity of peptides to form an aggregate at an organic solution/aqueous solution interface.

Peptides which are identified from the virtual screen above as being potentially capable of forming an aggregate at an organic solution/aqueous solution interface may be selected for optional subsequent experimental validation of such peptides being capable of forming emulsions of organic/aqueous mixtures.

Thus in a further aspect there is provided a method of virtually identifying a peptide as being capable of emulsion formation, the method comprising selecting a propensity of aggregation (AP*) at an organic solution/aqueous solution interface. The method may comprise initially selecting a peptide which displays a propensity of aggregation (AP) in aqueous solution and thereafter selecting a peptide for its propensity of aggregation (AP*) at an organic solution/aqueous solution interface.

The method may be carried out in two stages, such that the AP may first be determined and only peptides which show a suitable AP in aqueous solution are selected for determination of AP* at an organic solution/aqueous solution interface.

In addition—or alternatively—to AP determination, a hydrophilicity-adjusted measure of propensity for aggregation (AP_{H}) for the peptide may be determined, the AP_{H} being determined by adjusting a measure of propensity of aggregation (AP) for the peptide in dependence on a measure of hydrophilicity for the peptide.

By adjusting the AP in dependence on a measure of hydrophilicity, an improved measure of propensity for aggregation may be obtained. AP_{H} may be used to provide a method of virtual screening of peptides for their propensity to form aggregates in solution.

The measure of hydrophilicity may comprise a sum of Wimley-White whole-residue hydrophobicities for amino acids in the peptide or any similar hydrophobicity scale (such as the Kyte and DooLittle scale [Kyte J, Doolittle R F. J Mol Biol. 1982 May 5; 157(1):105-32], or the Hessa and Heijne scale [Hessa T, Kim H, Bihlmaier K, Lundin C, Boekel J, Andersson H, Nilsson I, White S H, von Heijne G. Nature. 2005 January 27; 433(7024):377-81]) that relates the hydrophobicity of amino acids to rank the relative hydrophobicity/hydrophilicity of the peptide.

Adjusting the AP for the peptide in dependence on a measure of hydrophilicity for that peptide may comprise raising the AP to a power and multiplying by the measure of hydrophilicity.

At least one of the AP and the measure of hydrophilcity may be normalised prior to the adjusting of the AP in dependence on the measure of hydrophilicity.

Determining AP_{H} for the peptide may comprise using the equation: AP_{H}=(AP′)^{α}(log P)′ wherein α is a numerical constant, log P is the measure of hydrophilicity for the peptide, and an apostrophe denotes normalisation. α may have a value between 0.5 and 5, optionally between 1 and 4, further optionally between 1 and 3.

Changing the value of a in the above equation may change the relative weighting between the AP and the measure of hydrophilicity. Where a value for AP_{H}, for example a threshold value for AP_{H}, is determined, the value for AP_{H} may be dependent on the value for a.

The peptide may comprise a tripeptide, tetrapeptide, pentapeptide or even larger peptide. Preferably the peptide is a tripeptide.

Identifying the AP*, AP and/or AP_{H} of a peptide may comprise determining whether the determined AP*, AP and/or AP_{H} for the peptide meets or exceeds a threshold value for AP*, AP and/or AP_{H}.

A threshold value may be determined for AP*/AP/AP_{H}, with peptides having a value of AP*/AP/AP_{H} meeting or exceeding the threshold being considered to have a high propensity for aggregation. Peptides with a high AP/AP_{H} value may be selected for determining AP*. Peptides with a high AP* may be selected to be synthesised. Thus, the method may further comprise synthesising peptides which display a AP* at an organic solution/aqueous solution interface and optionally testing such synthesised peptides for their ability to aggregate an organic solution/aqueous solution interface, or capability of forming an emulsion between an organic and an aqueous solution.

The peptide is one of a plurality of peptides, and the identifying of the peptide comprises determining an AP*/AP/AP_{H} for each of the plurality of peptides and identifying at least one of the plurality of peptides in dependence on the determined AP*/AP/AP_{H} for the plurality of peptides.

The plurality of peptides may comprise, for example, a plurality of tripeptides. The plurality of peptides comprises substantially all possible combination of peptides encoded by naturally occurring amino acids. For example, for tripeptides there are 8,000 peptides (20³ where 20 is the number of naturally occurring amino acids). The approach can conceptually be expanded to include non-natural amino acids, such as Citrulline, norvaline, etc., and N- and C-terminal protected amino acids.

A method for virtually screening peptides (for example, tripeptides) may comprise screening large numbers of peptides by calculating AP/AP_{H} for each peptide, and identifying peptides based on the calculated AP/AP_{H}. Identification of peptides by AP_{H} may, in some circumstances, provide more promising candidates for aggregation than identification by AP alone.

Identifying at least one of the plurality of peptides in dependence on the determined AP/AP_{H} for the plurality of peptides may comprise identifying a subset of the plurality of peptides, the subset comprising the peptides having the highest AP/AP_{H}.

The subset of the plurality of peptides may comprise a portion, such as the 100 peptides having the highest AP/AP_{H}, optionally the 200 peptides having the highest AP/AP_{H}, further optionally the 400 peptides having the highest AP/AP_{H}.

The subset of the plurality of peptides may comprise 10% of the plurality of peptides having the highest AP/AP_{H}, optionally the 5% of the plurality of peptides having the highest AP/AP_{H}, further optionally the 2% of the plurality of peptides having the highest AP/AP_{H}.

The number of peptides identified by AP/AP_{H} from the plurality of peptides may depend on the number of peptides in the plurality. For example, from the 8,000 possible tripeptides, a subset of 400 peptides (5% of all peptides) may be selected.

Identifying the subset of the plurality of peptides may comprise identifying all peptides in the plurality of peptides having an AP/AP_{H} greater than a threshold value for AP/AP_{H}, or greater than or equal to a threshold value for AP/AP_{H}.

The threshold value may be determined by calculating the AP/AP_{H} for each peptide in the plurality of peptides and setting the threshold value to deliver a desired size of subset. The threshold value may be determined based on knowledge of the AP/AP_{H} of peptides that have successfully aggregated.

The subset of peptides identified as having a high and/or above threshold AP/AP_{H} value may be selected for AP* determination.

The method may further comprise displaying the AP* for the peptide from simulation. Displaying the AP* for the peptide from simulation may comprise performing a molecular dynamics simulation for the peptide in an organic/aqueous solution mixture and displaying the results. An investigator may view the displayed results by way of a visual representation of aggregation formation being displayed on a monitor, such as a computer monitor, or the like. Alternatively, aggregation formation may be determined by way of image processing analysis software designed to identify simulated aggregates being formed at an organic solution/aqueous solution interface.

Where there are a plurality of peptides, the method may comprise obtaining the AP* for each of the plurality of peptides from a respective simulation, which may comprise a molecular dynamics simulation.

The AP/AP_{H}/AP* for a given peptide may comprise a ratio between a solvent accessible surface area at the beginning of the molecular dynamics simulation and a solvent accessible surface area at the end of the molecular dynamics simulation.

The above described methods may be further modified to take account of the measure of protonation of each amino acid individually and/or when part of a particular peptide sequence, such as a tripeptide. The amino and carboxy termini of peptides, as well as certain exposed amino acid side chains are capable switching between protonated and deprotonated forms, depending on the pH of the surroundings and the aggregation state and the inventors have observed that peptide aggregation or gelation can be affected by changes in pH. Therefore, screening for the ability of tripeptides to aggregate within different pH environments can be achieved by modifying the standard coarse-grained beads, which are parameterized for neutral pH, to represent the sidechains in the alternative protonation state. For example, rather than utilising a protonated N-terminus beads (NH₃⁺) a neutral N-terminus bead (NH₂) bead could be used to examine the effect of moving above pH 10, whereby the N-terminus would be deprotonated.

In a further aspect there is provided a method of producing a peptide aggregate comprising a peptide capable of self-aggregation at an organic solution/aqueous solution interface, the method comprising identifying a peptide by determining a measure of propensity of aggregation (AP) for the peptide in aqueous solution, optionally in dependence on a measure of hydrophilicity (AP_{H}) for the peptide, identifying by simulation whether or not the peptide is capable of self-aggregation at an organic solution/aqueous solution interface, and optionally synthesising the peptide.

The peptide may be one of a plurality of peptides, and the identifying of the peptide may comprise determining an AP/AP_{H} for each of the plurality of peptides and identifying at least one of the plurality of peptides for AP* determination and selection of a peptide which is capable of forming an self-aggregate at an organic solution/aqueous solution interface.

By observing the sequence of thus identified peptides, classes of peptides with certain self-assembly behaviours may be identified, for example those which contain for example, aromatic, anionic, cationic, H-bonding residues in certain positions.

In a further aspect there is a provided a peptide or nanostructure obtainable by any of the above methods.

Typically the peptides of the present invention may aggregate at an organic solution/aqueous solution interface, at a concentration of between 1-500 mM, such as 10-200 mM, 15-100 mM, or 20-60 mM.

The present inventors have observed that the amino acid composition of the peptides of the present invention and/or the modifications which can be made to the amino acid/peptides, can have an effect on the emulsion forming and/or stability properties of the emulsions. Thus, by altering the amino acid composition of the amino acid/peptides and/or the modifying groups, it is possible to alter the self-assembly properties of the peptides and consequent properties of the resulting emulsions. This can have an effect on, for example, the stability (towards variations in temperature, ionic strength, presence of certain small molecules, presence of solvents, exposure to pressure, shear, ultrasound or audible sound) of the emulsions, the ability to form emulsions with certain immiscible liquids, the ability of the liquids to form the dispersion or continuous phase, droplet size and/or critical emulsion concentration, sequence selective stabilisation of polymeric solutions, stabilisation of solutions containing biological macromolecules such as DNA, proteins, stabilisation of emulsions in biological fluids, inside cells, in tissues, responsiveness to light, pressure, presence of biomolecules, ionic strength, enzymes, ultrasound, magnetic field, electric field, or combinations thereof. Thus, by varying the amino acid, amino acid sequence and/or modifying groups, it is possible to effect emulsion properties. This can easily be tested by the skilled addressee by appropriate means and experimentation as taught herein and “Microemulsions: Properties and Applications (2008) CRC press and “Emulsions and emulsion stability: Surfactant Science Series/61” (2005) CRC press.

The amino acids/peptides of the present invention are understood to be provided at a sufficient concentration such that at the liquid-liquid interface they are able to interact with one another with sufficient strength to create a self-assembled fiber or structure which is able to form a network or film comprising many such fibers, spherical aggregates, tapes, 2D sheets or other nano-structures. Typically the amino acids/peptides are provided at a concentration of 1-50 mM, such as 5-25 mM, 7.5-15 mM, especially 10 mM. However, it is also possible to determine a suitable concentration for emulsion formation simply by testing varying concentrations of the peptide with chosen immiscible liquids and observing at what concentration emulsions may be formed. Thus, depending on the peptides and liquids being used, the concentration of the peptides for forming the emulsion may be outside of the above defined ranges.

The volume ratio of the first liquid to the second liquid is generally in the range of 1:20-20:1, more conveniently 1:10-10:1.

The term “self-assembled” or “self-assembly” is understood to mean that the amino acids/peptides are capable of undergoing spontaneous (or triggered by an applied stimulus, such as a change in pH, ionic strength, solvent polarity, light, sound, enzymatic action, catalysis) assembly into ordered fibers, tapes, spheres, sheets or related structures, typically with nanometer dimensions.

Conveniently the emulsions of the present invention can be formed without vigorous shaking or mixing. For example, the inventors have made emulsions according to the present invention simply by shaking by hand.

The peptide fibers or nano-structures may be formed from amino acids/peptides having the same amino acid sequence or mixtures of peptides having more than one different amino acid sequence. The amino acids/peptides may also form the fibers or other nano-structures in combination with other macromolecules, such as larger peptides or proteins. In some embodiments, the amino acids/peptides forming the fibers or structures have the same amino acid sequence and thus form a ‘homogeneous amino acid/peptide assembly’. In other embodiments two or more different amino acids/peptides form a ‘heterogeneous amino acid and/or peptide assembly’.

As used herein, the term “interface” refers to a surface forming the common boundary between two adjacent non-miscible liquids. A liquid-liquid interface is the surface forming the common boundary between two immiscible liquids, such as oil and water.

Advantageously, the emulsions of the present invention may be stable over long periods, which is distinguishable over emulsions formed with other surfactants such as SDS. In a preferred embodiment, the emulsions of the present invention, remain stable over a period of at least 1 week, 2 weeks, 4 weeks, 2 months, 6 months, 12 months or more when not in the presence of salt, for at least 12 h, 24 h, 2 days or more in the presence of 100 mM salt, such as a phosphate, chloride or thiocyanate, and/or a stable to exposure to heat, such as 50-70 C for 2-4 h, such as 60 C for 3 h. Alternatively, emulsions of the present invention may be stable at a first temperature, but may demulsify at a second temperature. For example, certain peptides maybe capable of forming emulsions at room temperature, but demulsify at elevated temperature, such as 40 C, 50 C, 60 C or higher.

Advantageously, the demulsification (i.e. breakdown of the emulsion) can be effected by the addition of one or more proteases, if required. For example, the emulsions of the present invention may be demulsified by the addition of the protease thermolysin. Alternative enzyme types that may be envisaged include esterases and phosphatases to change the amphiphathic balance of the peptide molecules. Other means of switching may also be envisaged, involving introduction of e.g. light switchable units that undergo conformational changes upon exposure to specific wavelengths, such as azobenzene. This could be incorporated at the N terminus or as side chain of an amino acid.

In a further aspect there is provided a method of making an emulsion, the method comprising mixing at least 2 substantially immiscible liquids in the presence of amino acids/peptides as described herein, in order to form an emulsion. The at least two immiscible liquids may be provided and the amino acids/peptides added thereto, or the amino acids/peptides may be added to one or more of said immiscible liquids before a further immiscible liquid or liquids is brought into contact with the immiscible liquid(s) containing the amino acids/peptides.

It will be appreciated that the amino acids/peptides must be provided at a concentration which is capable of allowing emulsion formation. Suitable concentration ranges and/or how to determine them are described herein.

Other optional method steps will be understood from the above description in relation to emulsion components and will not be repeated for brevity, but the skilled reader is directed to this.

The present invention may be used to provide emulsions which comprise an agent, such as a pharmaceutical agent or other agents identified herein above, which is otherwise only soluble or suitably dispersible in a oil or aqueous/water. For example, if a pharmaceutical or other agent is to be incorporated into an emulsion of the present invention, the agent may initially be included in an oil or organic phase before preparing the initial emulsion. In some embodiments, the pharmaceutical or other agent is soluble in the oil/organic phase. In other embodiments the pharmaceutical or other agent is insoluble in the oil/organic phase.

There is a need for pharmaceutical delivery carriers that are easy to prepare in the absence of non-pharmaceutical solvents, can carry a variety of pharmaceuticals, have appropriate pharmacokinetic properties including stability under biological conditions and/or deliver a pharmaceutical to a particular tissue or receptor. Additionally these pharmaceutical delivery vehicles should encapsulate or shield the pharmaceutical and deliver it in a concentrated fashion to the site of desired action, meanwhile masking it from immune clearance. There is a further need for pharmaceutical delivery carriers to deliver low amounts of pharmaceutical (e.g. antigenic protein) to specific cell types (e.g. dendritic cells) in a targeted fashion, in order to induce a sub-immunogenic activation of T cells. Alternatively, a concentrated bolus of pharmaceutical, for example a chemotherapeutic agent, should be delivered to cells again in a targeted fashion, so as to kill the target cell(s). The emulsions of the present invention may find application in this regard. Thus in a further embodiment, the present invention further provides an emulsion further comprising a pharmaceutical agent—this may include a small drug molecule, as well as nucleic acid, proteins, antibodies and antibody fragments and the like.

The emulsions of the present invention may also find application in the agricultural, food, cosmetics and/or catalysis fields. For example in the agricultural industry, emulsions are used as delivery vehicles for insecticides, fungicides and pesticides. These water insoluble biocides must be applied to crops at very low levels, usually by spraying through mechanical equipment. In cosmetics, emulsions are the delivery vehicle for many hair and skin conditioning agents.

Many food products are in the form of emulsions. Salad dressings, gravies and other sauces, whipped dessert toppings, peanut butter, and ice cream are also examples of emulsions of various edible fats and oils. In addition to affecting the physical form of food products, emulsions impact taste because emulsified oils coat the tongue, imparting a modified “mouth-feel” to the product.

In terms of emulsions which may find application in the food industry, emulsions are provided both in Ready To Use format such as UHT products, for example and in premix or concentrate forms such as Powder Premixes or Paste Concentrates, which when made up are effectively emulsions. Such emulsions may be stabilised by a variety of materials such as Modified (and Native) Starch, Stabilisers, Gums and emulsifiers (or combinations of).

The peptide emulsion formulations of the present invention have demonstrated gellation, emulsification, stabilisation and co-assembly properties which may allow replacement and/or a reduction of existing natural and un-natural materials employed in food products. Additionally, the peptide emulsion formulations of the present invention may provide unique properties which could offer new or unique textures which may be exploited in a variety of applications such as Desserts, Glazes or Sauces, Dairy Cream alternatives, Paste Ferments and icings/fillings or Cakes/finishing's, which may find application in chilled, and frozen products, as well as products which may be kept at (ambient) room temperature

It is envisaged that appropriately formulated food products which employ the emulsions of the present invention may display one or more of the following important technical/functional aspects:

Viscosity

Products require the correct viscosity dependant on application: Coating, cling, mouthfeel, texture, handling, application, processing.

Stability

Product stability is essential across various stages in manufacture/use:

Processing:

Stability to ensure homogenous product through all stages of processing. Destabilised products risk potential fall out of emulsion, build-up of material in plant with resultant product burn/blockage. Stable product in processing will enable:

Packing:

Consistent product for packing (at various temperatures for different products/processes: to ensure correct product attributes for end use)

Customer Use:

Consistent product and end product performance

Shelf Life:

Consumer expectation for homogeneous product: whilst separation can be evident without negative impact on product performance (Sterility, flavour, texture: organoleptic profile), appearance may influence user that there is a product issue with separation. Stability is there for essential.

Product Pack Format:

In UHT, for example, as the product format increases, the pressure and resultant forces on the emulsion become greater with separation becoming apparent more rapidly. This affects shelf life applied to the product format. 0.5-1 L products typically have 12 month shelf life, 10 L & 25 L typically have 9 months shelf life and 1000 L typically has 6 months shelf life.

Thermo Reversibility:

product viscosity changes at different temperatures. Viscosity control is one aspect derived from modified starch: the use of peptide formulations may deliver this control. A further area for development is desserts where a liquid gel is made possible via UHT processing and chilling a gel below its activation point. On reheat, this gel is then reactivated which will set when the end user chills it. Peptide formulations which gel in a similar fashion will offer similar products.

Raw Material Substitution

Modified (and Native) Starch, Stabilisers, Gums and emulsifiers (or combinations of) may be replaced with peptide emulsion formulations of the present invention.

Emulsification and Control of Solid Phase Products (Lecithin Effect in Chocolate/Paste Product Matrix)

Lecithin is essential in chocolate with regards to cost where the amount of Cocoa Butter can be reduced in the presence of Lecithin and still retain the necessary fluidity required for enrobing and moulding. Peptide formulations could potentially act as a functional replacement with surfactant properties. In Paste Concentrate Matrices, the functional emulsification properties may enable new paste format textures but would be more evident in finished products such as fermented goods (i.e. breads and rolls) where the emulsifier use ranges from film forming for bubble stability, regularity of bubble formation, texture of finished product, dough extensibility, dough conditioning, dough stability and shelf life.

Aeration, Bubble Formation or Foaming in Food and Non-Food (and Stability of)

Multiple materials are often required in Dairy Cream Alternatives (DCA's) to deliver high volume whip and stability performance. Peptide formulations may enable bubble formation and therefor foaming which could be used in both DCA's and non-food foams in other potential industries. The stability performance is expected to be high due to the functionality of the material that may be improved over existing complexes.

Stabilisation of Protein During Heat Treatment

The use of Peptides in combination with heat sensitive materials such as egg may act as a prohibitor for denaturation of protein on exposure to heat. Egg use in UHT formulations, for example, can be problematic due to denaturation and therefor viscosity build or particulate formation that can build up within processing.

Enhancement of Functional Properties of Materials

The use of peptides could facilitate in reduction in the use of existing high cost, highly functional materials such as by associating and/or acting as in combination with existing materials, e.g. Egg white association and performance ‘boost’. This would not be limited to egg white (e.g. potential enhancement of other materials such as milk proteins) and may be applied across multiple foodstuff applications.

Thermal/Process Conditions

Within the food industry multiple process conditions are used (often combinations of) for which the present invention will be compatible with. These include UHT to Pasteurisation process steps as well as minimal processing to deliver Fresh Ready to Eat products across all of the food industry product categories

### DETAILED DESCRIPTION

The present invention will now be further described by way of example and with reference to the Figures which show:—

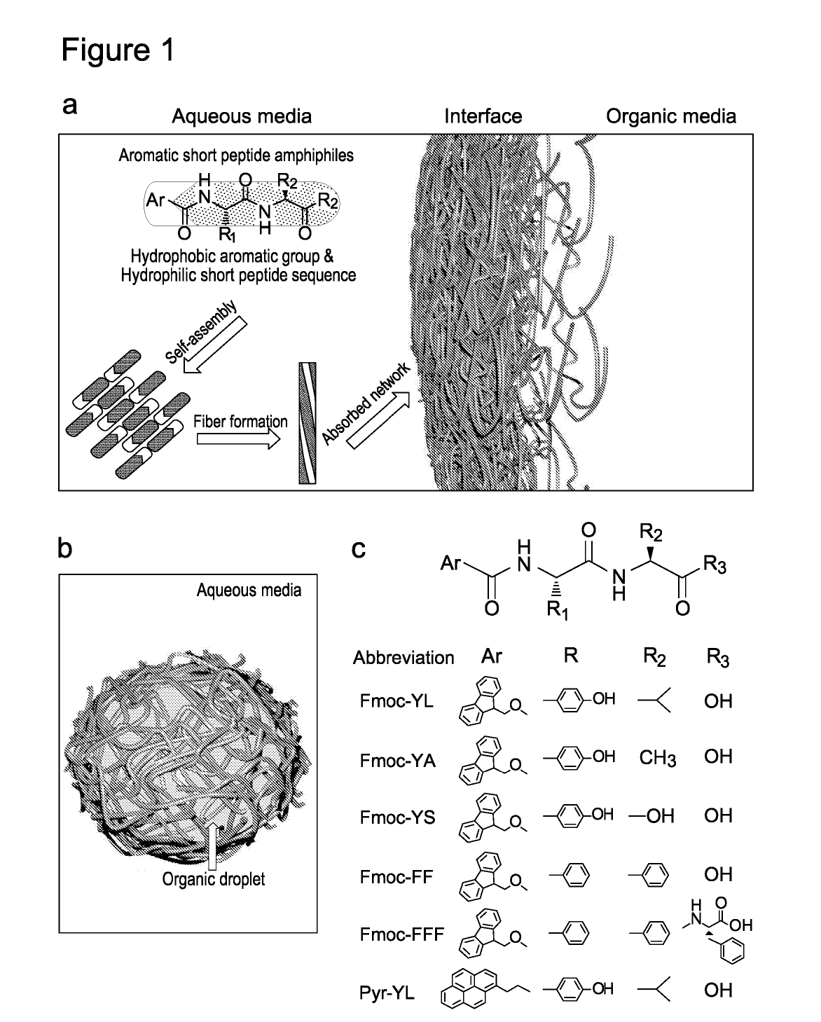

FIG. 1 shows (a) Cartoon of self-assembly and formation of fibrous network of aromatic short peptide amphiphiles at oil/water interface. (b) Cartoon of oil-in-water droplets stabilized by peptide fibrous network. (c) Chemical structure of aromatic peptide derivatives including Fmoc-YL, Fmoc-YA, Fmoc-YS, Fmoc-FF, Fmoc-FFF and Pyrene-YL.

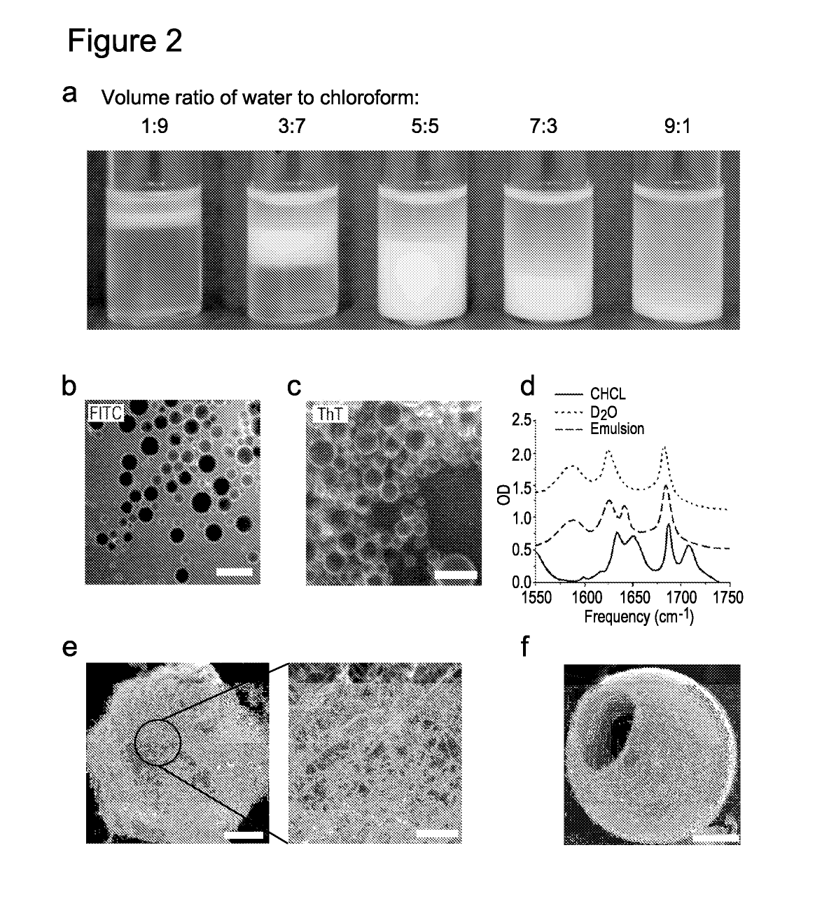

FIG. 2 shows (a) Optical photographs of glass vials in which chloroform-in-water emulsions (white foamy layer) were prepared by adding 10 mmol·L⁻¹ Fmoc-YL phosphate buffer solution (pH 8) to chloroform with manual agitation. From left to right, the volume ratio of buffer solution to chloroform is altered from 1:9, 3:7, 5:5, 7:3, to 9:1 and samples are named as W1C9, W3C7, W5C5, W7C3 and W9C1. (b) Fluorescent microscope image of chloroform-in-water emulsion droplets stabilized by Fmoc-YL networks containing FITC in water phase (Sample W3C7). Scale bar is 50 μm. (c) Fluorescent microscope image of chloroform-in-water emulsion droplets stabilized by ThT labelled Fmoc-YL networks (Sample W3C7). Scale bar is 50 μm. (d) FTIR spectra of self-assembly of Fmoc-YL in chloroform (black), D₂O phosphate buffer solution (pH 8) (red) and at interfaces stabilizing emulsions (blue). (e) SEM micrographs of Fmoc-YL networks at chloroform/water interface stabilizing the chloroform-in-water emulsions. The samples are prepared at freeze-drying condition. The scale bars are 50 μm (left) and 2 μm (right). (f) SEM micrograph of Fmoc-YL microcapsules at chloroform/water interface. The sample is prepared at air-drying condition and scale bar is 2 μm.

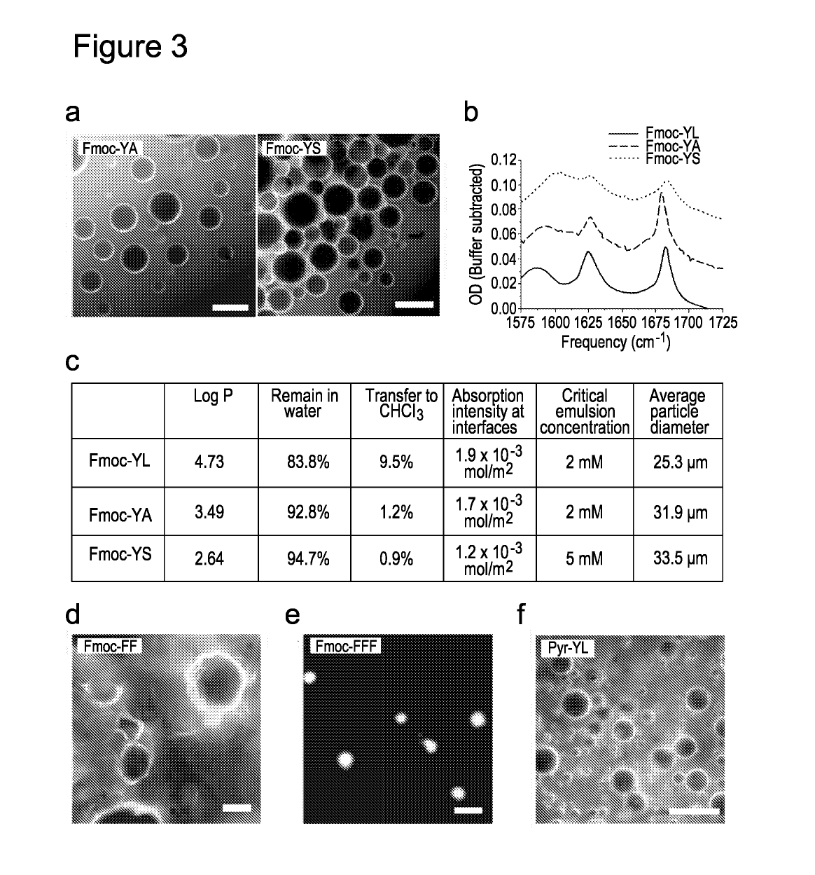

FIG. 3 shows (a) Fluorescent microscope images of chloroform-in-water emulsion droplets stabilized by Fmoc-YA (left) and Fmoc-YS (right) networks containing FITC in water phase. Scale bar is 50 μm. (b) FTIR spectra of 10 mmol·L⁻¹ Fmoc-YL, Fmoc-YA and Fmoc-YS in D₂O phosphate buffer solution (pH 8). (c) Table of the calculated partition coefficient (c Log P) and the measured partitioning of peptides between water, chloroform and accumulated at the interface, the critical emulsion concentration of Fmoc-YL, Fmoc-YA and Fmoc-YS and the average diameters of emulsions droplets. Fluorescent microscope images of (d) chloroform-in-water emulsion droplets stabilized by Fmoc-FF containing FITC in water phase, (e) water-in-chloroform emulsion droplets stabilized by Fmoc-FFF containing FITC in water phase and (f) chloroform-in-water emulsion droplets stabilized by Pyrene-YL. Scale bar is 50 μm.

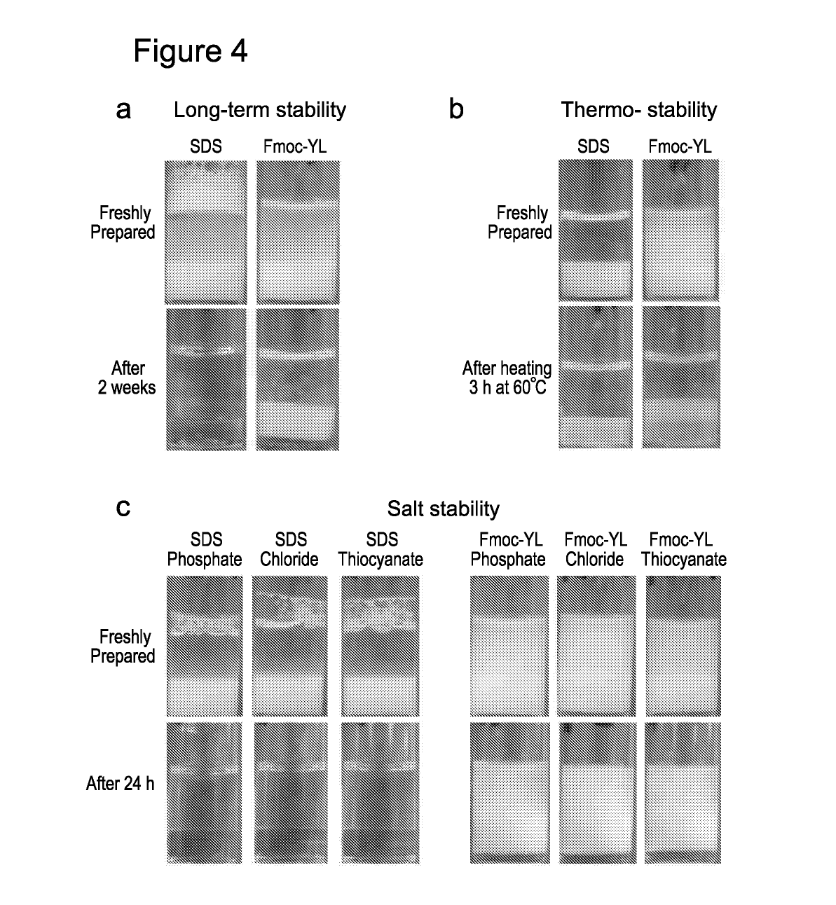

FIG. 4 shows (a) Optical photographs of glass vials in which chloroform-in-water emulsions (white foamy layer) were prepared with 10 mmol·L-1 SDS solution and Fmoc-YL phosphate buffer solution by manual agitation. The top images show freshly prepared emulsions and the bottom images show emulsions incubated for 2 weeks. (b) Optical photographs of glass vials in which chloroform-in-water emulsions (white foamy layer) were prepared with 10 mmol·L-1 SDS solution and Fmoc-YL phosphate buffer solution by manual agitation. The top images show freshly prepared emulsions and the bottom images show emulsions heated at 60° C. for 3 hours. (c) Optical photographs of glass vials in which chloroform-in-water emulsions (white foamy layer) were prepared with 10 mmol·L-1 SDS and Fmoc-YL in 100 mM phosphate, chloride and thiocyanate buffer solution by manual agitation. The top images show freshly prepared emulsions and the bottom images show emulsions incubated for 24 hours.

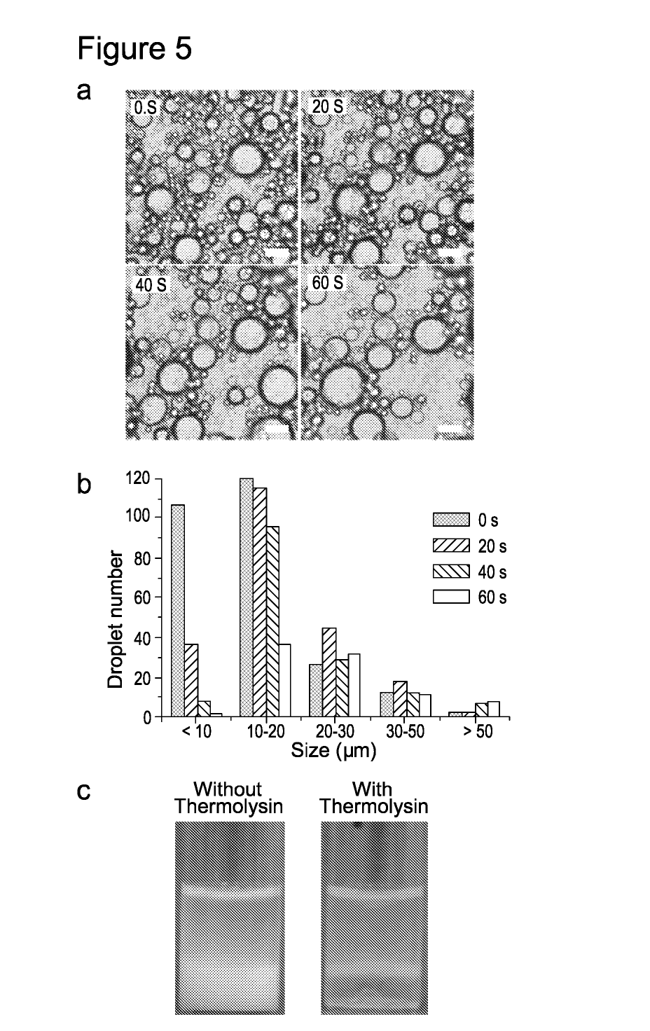

FIG. 5 shows (a) Optical microscope images of adding 1 mg·mL⁻¹ thermolysin buffer solution into chloroform-in-water emulsion droplets stabilized with 2 mmol·L⁻¹ Fmoc-YL buffer solution after 0, 20, 40, 60 seconds. Scale bar is 50 μm. (b) Histogram of the size distribution of adding 1 mg·mL⁻¹ thermolysin phosphate buffer solution into chloroform-in-water emulsion droplets stabilized with 2 mmol·L⁻¹ Fmoc-YL buffer solution after 0, 20, 40, 60 seconds. (c) Optical photographs of vials in which emulsions formed (left) and demulsified (right) in addition of thermolysin. Emulsions were stabilized with 2 mmol·L⁻¹ Fmoc-YL buffer solution in absence (left) and presence (right) of 1 mg·mL⁻¹ thermolysin after 10 min.

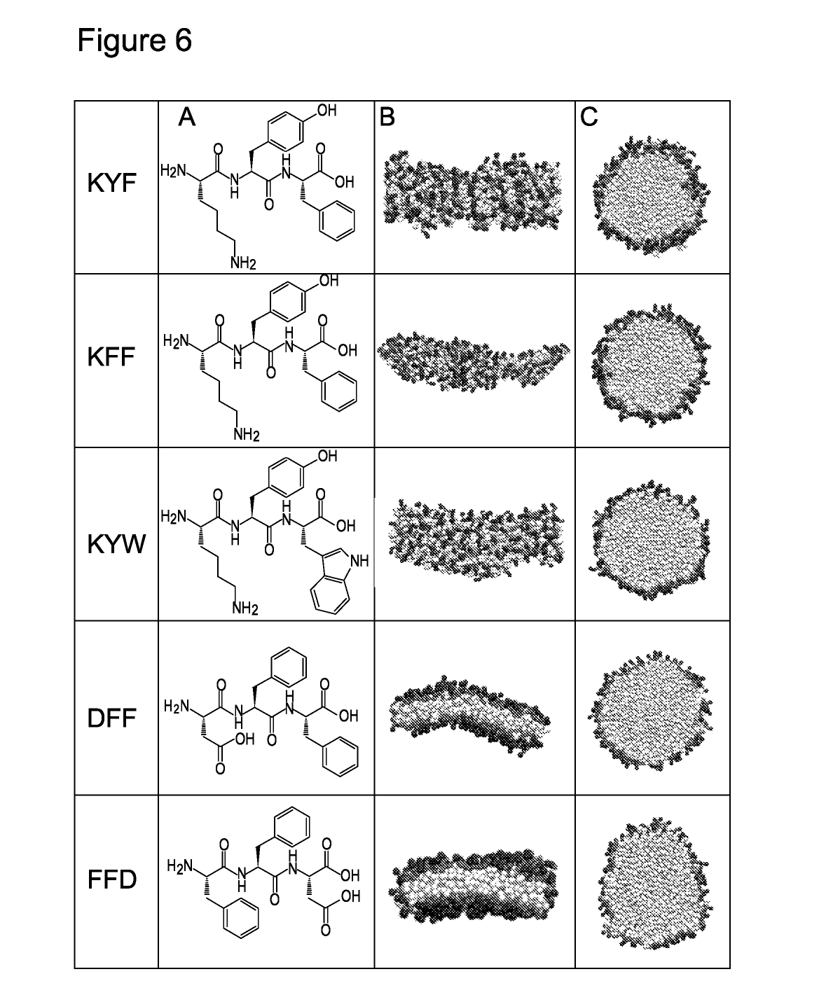

FIG. 6: Schematic for computational stabilized emulsions A) Tripeptides KYF/KFF/KYW/DFF/FFD B) Aqueous MD simulations showing self assembled nanostructures C) Self-assembled stabilized emulsions droplet. Simulation time 9.6 μs.

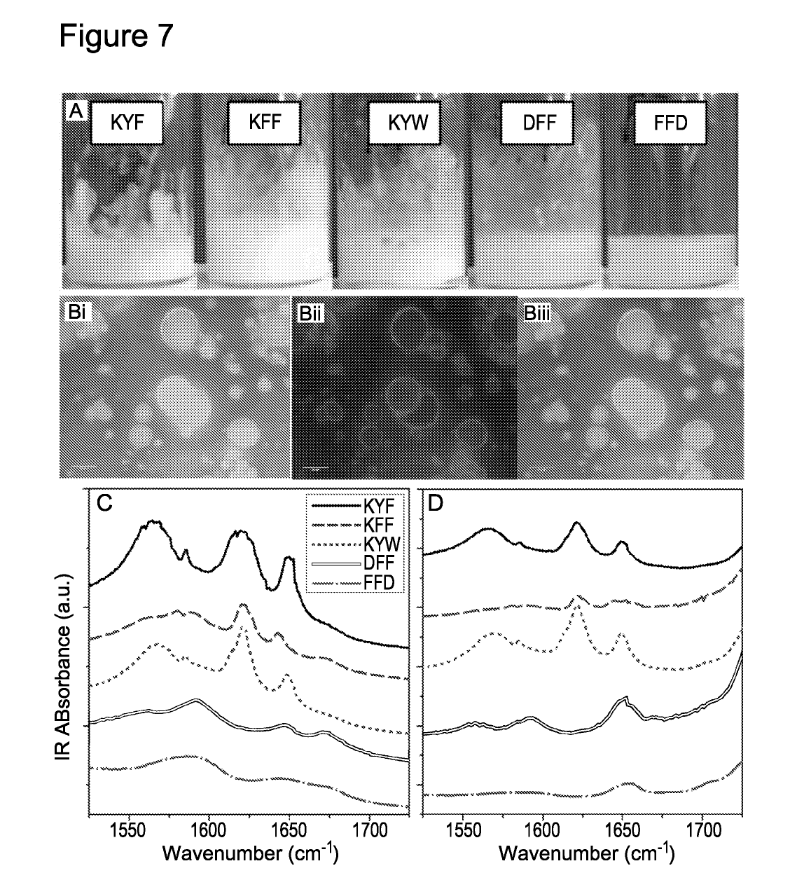

FIG. 7: Experimental observations of peptide emulsions A) Emulsions formed from each of the tripeptides B) Fluorescent microscope of KYW labeled with i) Sudan II ii) Thioflavin T iii) overlay of both, scale bar 10 μm C) FTIR of the samples in the aqueous state D) FTIR of samples in the emulsions state showing amide I region.

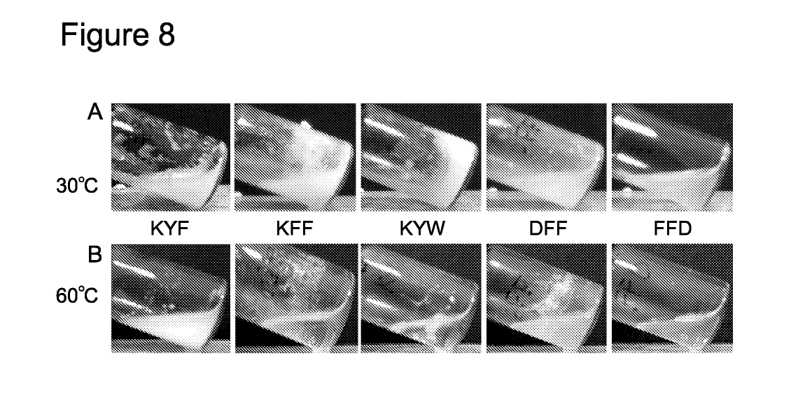

FIG. 8: Temperature effects on peptide emulsions at A) 30° C. B) 60° C.

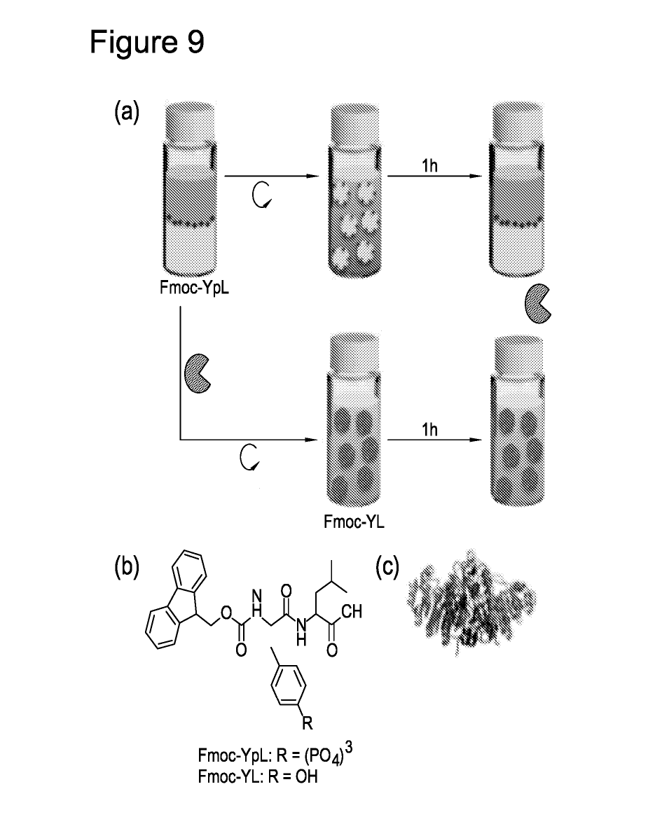

FIG. 9(a) Schematic representation of the behaviour of Fmoc-YpL before and after alkaline phosphatase dephosphorylation in a chloroform/water biphasic system, showing the ability of Fmoc-YL to stabilize emulsions, contrary to Fmoc-YpL which follows a surfactant-type behavior and relaxes back to two-phases after 1 hour. Cyan blue represents water, yellow chloroform and green the alkaline phosphatase; (b) Chemical structures of aromatic peptide amphiphiles Fmoc-YpL and Fmoc-YL; (c) Alkaline phosphatase structure.

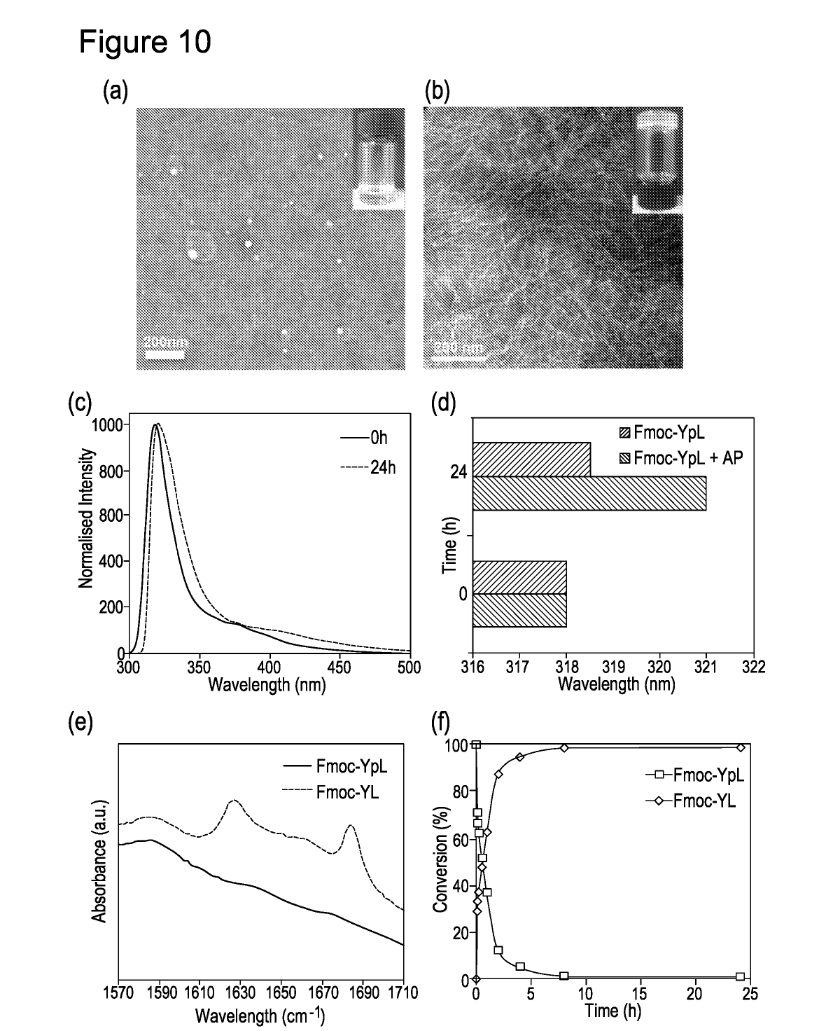

FIG. 10. Zoomed-in TEM image (original in Figure S1 from ESI) and macroscopic appearance of Fmoc-YpL (a) and TEM image of Fmoc-YL (b), showing the importance of the alkaline phosphatase to initiate self-assembly into a nanofibrous network and self-supporting hydrogel (Ammonium molybdate 2% stain); (c) Normalised fluorescence emission spectra of Fmoc-YpL (0 h) and Fmoc-YL achieved 24 h after enzyme addition (un-normalised data included in Figure S3 from ESI) (excitation 280 nm); (d) Representation of the lambda max wavelength at which fluorenyl peaks were observed before and 24 hours after enzyme addition, showing a redshift (except for the Fmoc-YpL control); (e) Amide region of FTIR absorbance of Fmoc-YpL and Fmoc-YL; (f) Dephosphorylation from Fmoc-YpL to Fmoc-YL monitored by reversed phase HPLC.

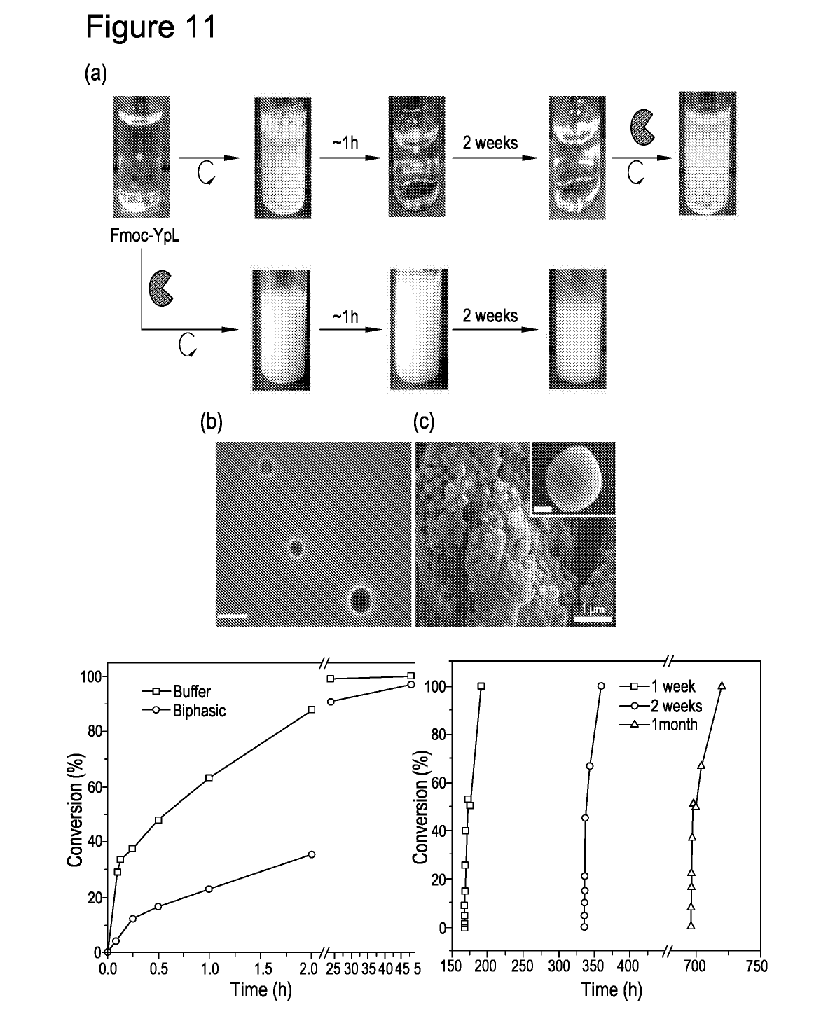

FIG. 11a) Optical photographs of glass vials showing the behaviour of Fmoc-YpL and Fmoc-YL in a chloroform/water biphasic system, immediately after hand shaking for 5 seconds and after 1 hour/2 weeks, in addition to the ability of Fmoc-YpL, completely demulsified after 2 weeks, to form an emulsion when alkaline phosphatase is added;

(b) Fluorescence microscopy image of chloroform-in-water emulsion stabilized by nanofibrous networks of Fmoc-YL containing FITC in water phase. Scale bar is 100 μm; (c) SEM image of chloroform-in-water emulsion droplet. Scale bar is 1 μm. Inset presents a zoomed-in chloroform-in-water droplet. Scale bar is 10 μm; (d) Dephosphorylation monitored by reversed phase HPLC in buffer and in the biphasic system, showing that alkaline phosphatase is active in the chloroform/water system; (e) Dephosphorylation monitored by reversed phase HPLC when adding alkaline phosphatase to the demulsified Fmoc-YpL at different timings.

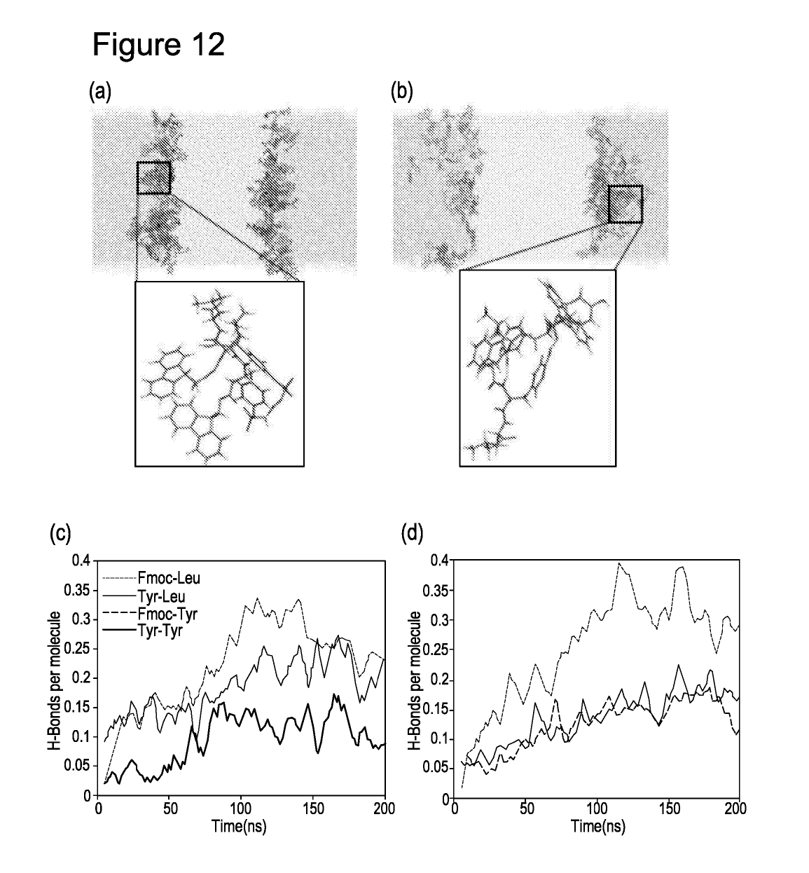

FIG. 12. (a) Snapshot of Fmoc-YpL system after 200 ns. Fmoc is represented in blue, Tyrosine and Leucine in red, phosphate group in black, ions in grey, water in red and octanol in cyan. Inset presents one Tyr-Tyr H-bonding between 2 molecules, and the surfactant-type behaviour, coloured by element; (b) Snapshot of Fmoc-YL system after 200 ns. Fmoc is represented in blue, Tyrosine and Leucine in red, water in red and octanol in cyan. Inset presents Fmoc-Leu and Fmoc-Tyr H-bonds between 2 molecules; c) Hydrogen bonds per molecule between Fmoc-YpL molecules throughout the simulation in biphasic system; (d) Hydrogen bonds per molecule between Fmoc-YL molecules throughout the simulation.

In this work, there is disclosed a series of aromatic short peptide amphiphiles which are exemplified by combining 9-fluorenylmethoxycarbonyl (Fmoc) or pyrene (Pyr) with di- or tri-peptides tyrosine-leucine (YL), tyrosine-alanine (YA), tyrosine-serine (YS), di-phenylalanine (FF) and tri-phenylalanine (FFF) with varying hydrophobicity and functional groups (FIG. 1) These peptide amphiphiles self-assemble at organic/aqueous interfaces rapidly forming a highly stable microcapsules fibrous network. Unlike absorption of traditional surfactants with hydrophilic head and hydrophobic tail at interfaces, the nanostructures self-assembled by aromatic π-π stacking and hydrogen bonding of peptide sequences to stabilize the organic (or water) droplets in aqueous (or organic) media (FIG. 1b).

Initial interest in studying aromatic peptide amphiphiles at the organic/aqueous interface started from the observation that aromatic peptide Fmoc-YL forms a gel in both phosphate buffer solution (10 mM) and chloroform (25 mM). At low concentrations Fmoc-YL (0.5 mM) was shown to transfer between aqueous and organic phases to reach an equilibrium distribution. By adding different volumes of chloroform to 10 mM Fmoc-YL buffer solution at 80° C. (the volume ratio of buffer solution to chloroform is altered from 1:9, 3:7, 5:5, 7:3, to 9:1), after manual agitation for 5 seconds emulsions form in vials, as FIG. 2a shown. Unexpectedly, these remain stable for months, showing that the formed peptide layers provide an excellent barrier to prevent coalescence. In the images shown, the milky layers are emulsions and the transparent layers above and below are the aqueous and chloroform phase, respectively. UV-Vis was used to determine the amount of Fmoc-YL that remained in water phase and transferred to chloroform. Fluorescein isothiocyanate (FITC) was used to label the aqueous phase in the emulsion layers for imaging by fluorescence microscopy. FIG. 2b indicates that chloroform-in-water emulsions form after emulsification stabilized by Fmoc-YL.

The absorption of Fmoc-YL at the chloroform/water interfaces could be quantified by UV-Vis spectra by measuring the concentration in each phase and with the remainder absorbed at the interface. Based on UV analysis, in a 50:50 water/chloroform system, the amount of Fmoc-YL absorbed at the chloroform/water interface could be calculated as 1.9 mmol·m⁻². The calculated maximum absorption of a close-packed monolayer of Fmoc-YL is 3.4 μmol·m⁻² indicating that Fmoc-YL absorbed at the interfaces is composed of a film rather than a monolayer.

The structure of this peptide interfacial film at the chloroform/water interface was investigated, using a range of microscopy and spectroscopy technologies. Thioflavin T (ThT) was used to label self-assembled peptide structures. After dissolving the Fmoc-YL with ThT in both solvents, there is low emission in water and almost no emission in chloroform is observed, while upon gelation (24 h) the appearance of stronger emission in both water and chloroform demonstrates that the self-assembled β sheet-like of fibrous structures are formed. ThT was subsequently used to label the interfacial film, FIG. 2c shows that a Fmoc-YL shell stabilized the organic droplets suggesting the self-assembly of peptide β sheet-like structures at the interface.

Infrared spectroscopy was then used to determine the H-bonding interactions that underpin self-assembly of Fmoc-YL fibrous structure in water, chloroform and at the interface. FIG. 2d shows an infrared absorption spectrum in D₂O typical for peptides in a well-ordered β sheet-like arrangement with peaks at 1623 and 1684 cm⁻¹ for amide and carbamate moieties, respectively. In chloroform, these peaks were observed at 1632 and 1687 cm⁻¹ with an additional absorption at 1652 cm⁻¹ indicating the presence of a less-ordered H-bonding network. Additionally, a peak assigned to the carboxylate group of the C-terminus was found at 1588 cm⁻¹ in water, while a peak was observed at 1708 cm⁻¹ in chloroform indicating protonation of the C-terminus in the organic solvent. At the interface, Fmoc-YL adopts a confirmation that is similar to the chloroform system with two peaks, indicating a less ordered β sheet-like environment (1623 and 1641 cm⁻¹). The C termini remain (in part) deprotonated as indicated by a peak at (1588 cm⁻¹) suggesting that the nanostructured network is predominantly situated in the aqueous phase.

A scanning electron microscope (SEM) was used to visualize the interfacial Fmoc-YL film. Freeze-drying was used to prepare the samples with retention of structure. FIG. 2e shows fibrous networks at the interface. Upon air-drying condition, FIG. 2f shows the peptide stabilized emulsion droplets that remain as microcapsules after solvent evaporation and consequent shrinking.

The properties of aromatic peptide amphiphiles, such as hydrophobicity and chemical groups which may affect the emulsification, can be altered by changing the aromatic group or peptide sequence. We prepared a series of peptide amphiphiles by changing one amino acid on the peptide sequence with decreasing hydrophobicity from tyrosine-leucine (YL), tyrosine-alanine (YA) to tyrosine-serine (YS). Fmoc-YA can form gel in both buffer solution and chloroform (30 mM), while Fmoc-YS only forms gel in aqueous media under these conditions. Atomic Force Microscopy (AFM) images show the formation of fibrous structure of Fmoc-YL, Fmoc-YA and Fmoc-YS gels in buffer solution to demonstrate their propensity for unidirectional assembly. FIG. 3a shows that Fmoc-YA and Fmoc-YS can also stabilize chloroform-in-water emulsions. Infrared spectra (FIG. 3b) confirm the presence of β sheet-like H-bonding in Fmoc-YL and Fmoc-YA in aqueous media with a much weaker contribution for Fmoc-YS. FIG. 3c lists the calculated partition coefficient (c Log P) and the measured partitioning of peptides between water, chloroform and accumulated at the interface. These values correlate with the critical emulsion concentration (obtained from emulsification experiments) of Fmoc-YL, Fmoc-YA and Fmoc-YS and the average diameters of emulsions droplets. It demonstrates that more hydrophobic peptide amphiphiles and stronger H-bonding interactions between the peptide backbones lead to stronger absorption at the chloroform/water interfaces resulting in a decrease in size of emulsion droplets and lower critical emulsion concentrations.

To further increase the hydrophobicity, di- and tri-phenylalanine were tested as the peptide sequences (Fmoc-FF and Fmoc-FFF). Fmoc-FF was previously demonstrated to form nanofibrous structures (REF). FIG. 3d shows that self-assembled fibres labelled with FITC stabilize the chloroform droplets at the interface. The Fmoc-FFF amphiphiles become too hydrophobic to dissolve in the water phase, but they do dissolve in chloroform. Upon preparation of 10 mM Fmoc-FFF chloroform solution and added to buffer solution containing FITC and emulsification, the core of the emulsion droplets are fluorescent which indicates the formation of water-in-chloroform emulsions (FIG. 3e). Replacing the Fmoc group with pyrene, amphiphiles and the emulsion systems are inherently endowed with fluorescent properties. FIG. 3f shows the formation of chloroform-in-water emulsion with blue emission stabilized by self-assembly of Pyrene-YL. There is the clear expectation, based upon the results presented that it should be possible to change the peptide sequences and aromatic moieties to further to optimize and functionalize the emulsion systems.

The attraction of using self-assembled barriers of aromatic peptides amphiphiles to stabilize emulsions rather than traditional surfactants, e.g. sodium dodecyl sulfate (SDS), dramatically enhances the stability of emulsions. FIG. 4a shows that after leaving them for two weeks at room temperature, the emulsions stabilized by SDS are demulsified and phase-separated, while the emulsions stabilized by Fmoc-YL are still stable. Fmoc-YL stabilized emulsions are heat stable with no visible change observed after exposure to 60° C. for 3 h, which is comparable to the performance of SDS (FIG. 4b). In 100 mM phosphate, chloride and thiocyanate solution, 10 mM SDS stabilized emulsions are phase-separated after 24 h as FIG. 4c shows, while Fmoc-YL networks are not influenced by the salt effect. The inventors have observed that Fmoc-YL can also stabilize both hexadecane-in-water and mineral oil-in-water emulsions instead of chloroform making the approach generally applicable for variety of organic media.

Another vital advantage of using peptide self-assembly to stabilize the emulsion systems is the ability to digest the stabilizing film using a suitable enzyme. Proteases, the enzymes that cleave peptide bonds cause disassembly of amphiphiles. FIGS. 5a and 5b show that after adding thermolysin to Fmoc-YL stabilized emulsions, the emulsion droplets are demulsified and coalesce to bigger droplets in 60 seconds. When the conversion of cleaving reaction, catalyzed by thermolysin in aqueous media, reaches 50%, detected by high-performance liquid chromatography (HPLC) after 10 minutes, the complete demulsification and phase separation was observed FIG. 5c.

The computational screening protocol, reported previously,¹⁻² was applied to identify tripeptides that were able to self-assemble in water. From this initial screen of approximately 8000, a subset of tripeptides were identified that showed the potential to form fibers and bilayer structures, which was considered as a pre-requisite for the compounds to act as emulsifiers. This process led to the selection of five tripeptides (FIG. 6A) that were simulated, using the MARTINI coarse-grained force field,³ for a further 9.6 μs in both water and water/octane solutions

KYW, KFF and KYF have been previously identified as tripeptides that are able to form hydrogels. The simulation of these peptides in water results in extended fibril structures (FIG. 6B), which is indicative of self-assembled fibers. However, two additional peptides were identified: FFD and DFF, which after further simulation in water, were shown to self-assemble into a bilayer-like structure (FIG. 6B). Since this new structure shows potential amphiphilic behavior, this suggests it may be a strong candidate for forming emulsions.

The simulation of the tripeptides in a biphasic system was modeled through the use of an octane/water solvent box. Each of the tripeptides were then subjected to a new 9.6 μs simulation in the biphasic system to determine whether they would be able to stabilize the octane within the aqueous solution. The simulations show the assembly of the organic solvent as droplets with the peptides assembled at the water/octane interface. As expected, the peptides assemble with the hydrophobic groups exposed to the organic core of the droplet thus decreasing the interfacial tension between the two phases. Similarly, the hydrophilic groups act as a barrier for the water phase. The arrangement of the peptides in such an assembly indicates the peptides act as amphiphiles stabilizing the interface. The inability of the tripeptides to form nanofibers around the interface is related to the size limitations of the model. The simulation involves 300 tripeptides, which does not provide sufficient coverage, when self-assembled into a fiber, to encapsulate the octane droplet. Nonetheless, the ability of the tripeptides to interact with both the organic and aqueous phases is considered as a positive indicator for these molecules to act as emulsifiers and therefore laboratory experiments were carried out to test this prediction.

The five tripeptides were purchased at >98% purity. Each of the tripeptides were then dissolved in water and the pH was altered to a neutral pH ˜7.5. To create the emulsions, 100 μL of sudan II labeled rapeseed oil was added to each of the systems. Rapeseed oil was chosen for comparison with food regulated oils. Homogenization was carried out on each sample for 5 secs thereafter; the samples were stored for 24 hrs to ensure a stable emulsion was formed. Visual inspection of the resulting emulsions revealed a variety of stabilities across the five tripeptides. KYF, KFF and KYW form more stabilized emulsions than DFF and FFD, which suggests that the ability of these tripeptides to form nanofibres, as observed in the aqueous state, may play a pivotal role in the stability of the emulsion. The opacity of the samples (FIG. 7A) differs between the surfactant like (FFD and DFF) and fibrous (KYF, KFF, KFW) emulsifiers, with the more opaque emulsion indicating greater stability due to the complete dispersion of oil within the aqueous phase. The inventors have also observed that emulsions can be formed using other oils, such as vegetable oil in combination with the peptides identified.

Fluorescent microscopy was carried out on each of the samples to identify the size and distribution of the droplets as well as to identify how the peptides interact at the interface. Labeling the organic phase with Sudan II revealed a mixture containing stabilized organic droplets. The introduction of Thioflavin T,⁴ which labels the peptide region (β-sheet formation) shows that the KYW is localized to the interface of the droplets. This suggests that, in the case of KYW, the tripeptide is self-assembling into fibrils are at the interface to create a network which is stabilizing the droplet, as observed herein for a range of Fmoc-dipeptides. The combination of the two dyes further highlights the localization of the tripeptide to the surface of the droplet, with negligible sensitivity away from the interfacial region.

To confirm that the tripeptides form self-assembled nanoscale network structures, rather than aggregating in a surfactant-like manner at the interface, FTIR spectroscopy was carried out to identify key interactions that are indicative of self-assembly of the peptides. To characterize the structures, studies were initially performed on the tripeptides in the aqueous phase, where KYF, KFF, and KFW are known to self-assemble and FFD and DFF do not (FIG. 7C). Significant changes are observed in the infrared spectroscopy upon aggregation of the peptides into nanostructures. Primarily, intense IR peaks around 1625 cm⁻¹ and 1650 cm⁻¹ show strong hydrogen bonding between the amide groups of the peptide chains, which are present in the fiber forming peptides. Similarly, a larger broad peak around 1560 cm⁻¹ is indicative of the deprotonated carboxylate group, COO⁻. The shift and broadening of this peak from the solution state to the gel state indicates an introduction of a salt bridge between either the corresponding termini or side group. This proposes a head to tail interaction between the peptides with hydrogen bonding between the two the self-assembled structures giving an overall extended stable structure. In the biphasic systems where the emulsions are formed, the samples that form fibers (KYF, KFF, KYW) have identical FTIR spectra to that observed in the aqueous phase. This indicates that similar fibrous networks are forming and therefore, these droplets are most likely stabilized by nanofiber networks. In contrast, comparison of the FTIR spectra for FFD and DFF between the aqueous and biphasic systems, reveal the emergence of peaks in the emulsion state indicating the formation of nanostructures, which stabilize the droplets. These peaks correspond to the C═O stretch of the peptide backbone. Since this is not observed in the aqueous state, this suggests that the introduction of the oil induces the self-assembling process for these peptides. Although, the peak is relatively weak, the single peak could show the parallel arrangement of the peptides, which are assembling in a similar manner to the traditional surfactant model.

The ability to trigger the separation of an emulsion through environmental triggers is useful property within a variety of application areas.⁵ In particular, the ability to degrade emulsions at various temperatures is a key property of interest for the application of emulsions in the food industry.⁶ Therefore, the thermal stability of the emulsions was investigated for each of the five tripeptides. Each sample was placed in an oil bath and the emulsions were monitored in 10° C. intervals (FIG. 8).

As the temperature is increased the emulsion separates into two different layers. This de-emulsification is observed for all samples at 60° C. apart from KYF, which remains in the emulsified state. This shows that KYF has a higher tolerance for heat than the other samples and that there are opportunities to tune heat resistance by sequence. Across the range in temperatures, it is clear that DFF and FFD de-emulsify relatively quickly which correlates with the initial observations that these peptides are not as strong emulsifiers, but also that traditional surfactants have thermal stability issues. KFF begins to breakdown at approximately 40° C. with KYW breaking down at 50° C. suggesting the strength of the hydrogel is directly proportional to the stability of the emulsion.

Switchable or stimuli-responsive surfactant ability is attractive for emulsion (de)activation at specific industrial process stages. By applying a specific trigger, the control of the emulsifying ability is enabled, being this a rapid efficient method of creating/breaking emulsions at a desired stage.

We studied the enzymatic conversion, using alkaline phosphatase, of the precursor Fmoc-tyrosine phosphate-leucine (Fmoc-YpL, FIG. 9b) into Fmoc-tyrosine-leucine (Fmoc-YL, FIG. 1b) in the aqueous phase. Having established the ability of the enzyme to trigger the self-assembly process, the second part of the experiment was to investigate the on-demand formation of amphiphile Fmoc-YL fibres at the chloroform/water interface, converting the surfactant-adsorbed biphasic mixture into a network-stabilized emulsion (FIG. 9a).

Results and Discussion

Enzymatic Conversion of Fmoc-YpL to Fmoc-YL. The precursor solution, 10 mM Fmoc-YpL in 0.6 M sodium phosphate buffer pH 8, is not able to form a gel and does not show any evidence of fibre formation (FIG. 10a) due to electrostatic repulsion between deprotonated phosphate groups. Upon addition of alkaline phosphatase, the clear solution of the precursor Fmoc-YpL was converted into Fmoc-YL, producing a nanofibrous network (FIG. 10b) that resulted in a hydrogel. Monitoring of the dephosphorylation reaction by reversed phase HPLC (FIG. 10f) revealed that approximately 90% of Fmoc-YpL is converted into Fmoc-YL after 2 hours, with complete conversion to Fmoc-YL achieved within 24 hours (FIG. 10f).

Enzymatically Triggered Emulsions.

When chloroform is added in a 1:1 volume ratio to the 5 mM Fmoc-YpL buffer solution and hand-shaken for 5 seconds, an emulsion is formed. Fmoc-YpL follows a surfactant-like behaviour, visible by the formed foam, where the amphiphile absorbs to the oil/water interface. However, the structure of Fmoc-YpL implies an inability to effectively stabilize the interface and demulsification occurs after one hour (FIG. 4a). On the other hand, when alkaline phosphatase is added to the biphasic system containing Fmoc-YpL and hand-shaken, nanostructures of Fmoc-YL are self-assembled at the interface between water and chloroform, creating an emulsion that is stable for months (FIG. 11a), as demonstrated previously. Enzyme-triggered self-assembly allows temporal control over emulsification, achieved by adding alkaline phosphatase to the Fmoc-YpL biphasic system (FIG. 11a).

TABLE 1
Partitioning of peptides (Fmoc-YpL and Fmoc-YL)
between water, chloroform and that remains at the
water/chloroform interface, along with logP values
of each determined by ChemDraw

| | Remain in | Transfer to | Transfer to | |
|---|---|---|---|---|
| | water | chloroform | interface | |
| | (%) | (%) | (%) | LogP |
| Fmoc-YpL | 31.03 | 0.74 | 68.23 | 5.67 |
| Fmoc-YL | 14.01 | 3.45 | 82.53 | 4.73 |

Molecular dynamic (MD) simulations were carried out to investigate the ability of Fmoc-YpL and Fmoc-YL to form ordered structures in a biphasic environment. The 60 molecules of Fmoc-YpL or Fmoc-YL were randomly distributed in the water phase of a large box which contained an TIP3P water and octanol. The tendency of both Fmoc-YpL and Fmoc-YL to aggregate towards the interface of the solvents was observed in the simulations (see final snapshots of the 200 ns simulation in FIGS. 12a and 12b). From the final snapshots of the system it is clear that although both systems are able to assemble at the interface of the solvents, Fmoc-YpL is evenly distributed along the length of the box, with minimal penetration into the octanol solvent, occurring predominantly for the Fmoc residues and the Leu residues (FIG. 6a). In contrast, Fmoc-YL is able to form a more ordered aggregate which allows partitioning of the resulting fibre-like structure into the octanol phase (FIG. 12b), which is consistent with the results from partitioning experiments (Table 1).

Conclusion

In conclusion, we demonstrate the use of short peptides, optionally containing aromatic groups, to self-assemble into nanofibrous networks at the organic/aqueous interface to emulsify and stabilize a variety of oil-in-water or water-in-oil emulsions. The formation of peptide microcapsules at interfaces provides long-term and higher stability against temperature and varied salts, compared with traditional surfactants, e.g. SDS. The peptide amphiphiles can, in some embodiments, be designed by altering aromatic moieties and peptide sequences to manipulate the emulsion stability, droplet size and/or critical emulsion concentration. Certain peptide microcapsules can disassemble in presence of proteolytic enzymes enabling on-demand demulsification under physiological conditions, or in response to elevated temperature. It is expected that through appropriate molecular design to include fully biocompatible analogues, by replacing the aromatic components with biocompatible ligands, such as nucleotides or other suitable groups, or using unmodified self-assembling aromatic peptides, as described. The interfacial networks presented here facilitate encapsulation and compartmentalization with potential applications in for example drug delivery and release, and the food industry.

We report the first unprotected tripeptides capable of self-assembling in biphasic systems to stabilize emulsions. These emulsions have shown a range of stabilities at both ambient and elevated temperatures giving a range of properties that are tunable and dependent on sequence. In particular, we show that fibrillar structures form more stable emulsions compared with the traditional surfactant model.

We have demonstrated the possibility of using aromatic dipeptide amphiphiles to enzymatically self-assemble at organic/aqueous interfaces to stabilize chloroform-in-water emulsions. We have demonstrated that Fmoc-YL is able to self-assemble in water following its enzymatic generation from the non-assembling precursor Fmoc-YpL. The self-assembled Fmoc-YL was shown to form nanofibres through non-covalent interactions, including π-stacking and H-bonding. When in a biphasic system, enzymatically-triggered Fmoc-YL self-assembles into nanofibrous networks at the chloroform/water interface, stabilizing the chloroform-in-water droplets and generating emulsions, which are stable for months. The stability of the emulsions and the possibility of switching on the emulsifier ability by adding the enzyme at different timings provides an extremely promising tool for several applications in chemical and other processes requiring emulsion formation.

Materials and Methods

Materials.

Fmoc-Tyr (97%), H-Leu-OtBu.HCl (≧98.0%), Ala-OtBu.HCl (≧98.0%), 0-tert-Butyl-L-Ser-tert-Butyl ester hydrochloride (≧98.0%), N,N-Diisopropylethylamine (DIPEA) (>99.5%), Trifluoroacetic acid (99%), Sodium hydroxide, 1-Pyreneacetic acid, Fluorescein isothiocyanate isomer I (≧90%), Thioflavin T, Sodium phosphate monobasic monohydrate (ACS reagent, 98.0%-102.0%), Sodium phosphate dibasic heptahydrate (ACS reagent, 98.0%-102.0%), Hexadecane (99%) and Mineral oil were purchased from Sigma-Aldrich and used as received. Fmoc-Phe-Phe-Phe-OH was purchased from BACHEM. 2-(1H-Benzotriazole-1-yl)-1,1,3,3-tetramethyluronium hexafluorophosphate (HBTU) was purchased from Novabiochem. Phosphate buffer solution (pH 8) was prepared by dissolving 94 mg NaH₂PO₄.H₂O and 2.5 g Na₂HPO₄.7H₂O in 100 ml water.

Fmoc-Tyr(PO(NMe₂)₂-OH (537.55 g·mol⁻¹) was purchased from Novabiochem. Fmoc-Tyr-OH (403.43 g·mol⁻¹), L-Leucine tert-butyl hydrochloride (223.74 g·mol⁻¹) and alkaline phosphatase from bovine, expressed in Pichia pastoris (5000 U·mg⁻¹ₚᵣₒₜₑᵢₙ, 20 mgₚᵣₒₜₑᵢₙ·mL⁻¹, 0.049 mL, Apparent molar weight 160 kDa) were supplied by Sigma Aldrich. One enzyme unit corresponds to the quantity of alkaline phosphatase hydrolysing 1 μmol of 4-nitrophenyl phosphate per minute at pH 9.8 and 37° C.

Synthesis of Modified Peptides

Synthesis of Fmoc-YL-OH:

Fmoc-Tyr-OH (1 g, 2.48 mmol), H-Leu-OtBu.HCl (0.663 g, 2.98 mmol) and HBTU (0.941 g, 2.98 mmol) were dissolved in anhydrous dimethylformamide (˜15 ml) with addition of DIPEA (1.08 mL, 6.2 mmol). The mixture was stirred for 24 hours. Product precipitated by adding saturated sodium bicarbonate solution (˜30 mL) and was extracted into ethyl acetate (˜50 mL). The mixture was washed with equal volume of saturated brine, 1M hydrochloric acid and brine. The resulting organic layer was dried by anhydrous magnesium sulphate and the ethyl acetate was removed by evaporation in vacuum. The resulting solid was then purified by column chromatography by using 2.5% methanol in dichloromethane as eluent. Fractions were tested using TLC under UV (254 nm) light to visualize the spots. Fractions containing the compound were combined and solvent removed in vacuum. The removal of the t-Bu was carried out by dissolving the sample in dichloromethane and adding 10 mL of trifluoroacetic acid. The mixture was stirred for 24 hours. The dichloromethane was removed by evaporation in vacuum and the TFA was removed with toluene (˜10 mL) and THF (˜2 mL). Solvent was removed by evaporation in vacuum (carried out in triplicate). The resulting solid was washed 6 times with cold diethyl ether and the product dried under vacuum.

The synthesis of the other Fmoc-dipeptides (Fmoc-YA-OH and Fmoc-YS-OH) follows the same experimental procedure as Fmoc-YL.

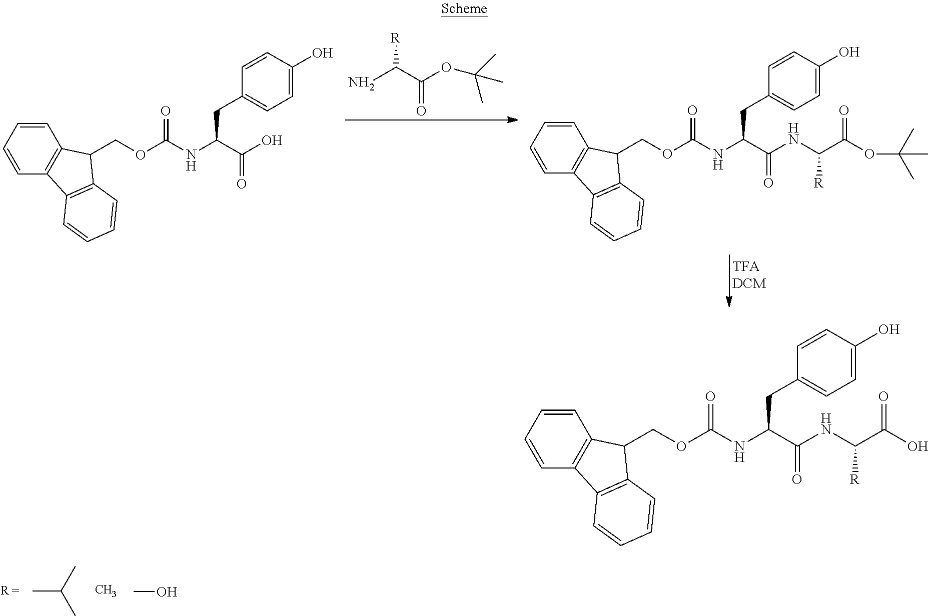

Fmoc-YL-OH

Purity by HPLC (214 nm)=97.00%. δ_{H} (DMSO, 500 MHz): 12.6 (1H, s, OH), 9.2 (1H, s, Tyr OH), 8.34-8.32 (1H, NH d, J=8 Hz), 7.89-7.87 (2H, d, J=7.5 Hz, 2 fluorenyl Ar-CH), 7.66-7.62 (2H, m, 2 fluorenyl Ar-CH), 7.55-7.53 (1H, d, J=9 Hz, NH), 7.43-7.39 (2H, m, 2 fluorenyl Ar-CH), 7.34-7.27 (2H, m, 2 fluorenyl Ar-CH), 7.10-7.09 (2H, d, j=8.3 Hz, 2 Tyr Ar-CH), 6.64-6.62 (2H, d, J=8.35 Hz, 2 Tyr Ar-CH), 4.25-4.10 (5H, m, fluorenyl CH, fluorenyl CH₂ and C_{α}H), 2.92-2.91 (1H, m, Tyr CH), 2.73-2.71 (1H, m, Tyr CH), 1.68-1.62 (1H, m, Leu CH) 1.55-1.52 (2H, m Leu CH₂), 0.9-0.83 (6H, m Leu 2CH₃) MS (ES+): m/z 517.2, [M+H]⁺.

Fmoc-YA-OH

Purity by HPLC (214 nm)=99.00%. δ_{H} (DMSO, 500 MHz): 12.5 (1H, s, OH), 9.1 (1H, s, Tyr OH), 8.28-8.26 (1H, d, J=8 Hz, NH), 7.88-7.87 (2H, d, J=7.5 Hz, 2 fluorenyl Ar-CH), 7.65-7.61 (2H, m, 2 fluorenyl Ar-CH), 7.53-7.51 (1H, d, J=9 Hz, NH), 7.43-7.39 (2H, m, 2 fluorenyl Ar-CH), 7.34-7.28 (2H, m, 2 fluorenyl Ar-CH), 7.10-7.09 (2H, d, j=8.3 Hz, 2 Tyr Ar-CH), 6.64-6.62 (2H, d, J=8.35 Hz, 2 Tyr Ar-CH), 4.19-4.10 (5H, m, fluorenyl CH, fluorenyl CH₂ and C_{α}H), 2.92-2.91 (1H, m, Tyr CH), 2.73-2.71 (1H, m, 1Tyr CH), 1.33-1.29 (3H, Ala CH₃) MS (ES+): m/z 475.07, [M+H]⁺.

Fmoc-YS-OH

Purity by HPLC (214 nm)=97.5.00%. δ_{H} (DMSO, 500 MHz): 12.5 (1H, s, OH), 9.1 (1H, s, Tyr OH), 8.28-8.26 (1H, d, J=8 Hz, NH), 7.88-7.87 (2H, d, J=7.5 Hz, 2 fluorenyl Ar-CH), 7.65-7.61 (2H, m, 2 fluorenyl Ar-CH), 7.53-7.51 (1H, d, J=9 Hz, NH), 7.43-7.39 (2H, m, 2 fluorenyl Ar-CH), 7.34-7.28 (2H, m, 2 fluorenyl Ar-CH), 7.10-7.09 (2H, d, j=8.3 Hz, 2 Tyr Ar-CH), 6.64-6.62 (2H, d, J=8.35 Hz, 2 Tyr Ar-CH), 4.7-4.6 (1H, m C_{α}H) 4.19-4.10 (4H, m, fluorenyl CH, fluorenyl CH₂ and C_{α}H), 2.92-2.91 (3H, m, 1Tyr CH, Ser CH₂), 2.73-2.71 (1H, m, Tyr CH). MS (ES+): m/z 491.00 [M+H]⁺.

Synthesis of Pyr-YL-OH:

1-Pyreneacetic acid (0.50 g, 1.92 mmol), L-Tyrosine tert-butyl ester (0.46 g 1.93 mmol) and HBTU (0.740, 1.95 mmol) were mixed in 10 mL dry DMF. 0.89 mL (4.8 mmol) of DIPEA was added to this solution and the mixture was stirred overnight under nitrogen atmosphere. After reaction, the product was extracted by 50 mL of ethyl acetate after successive wash with 20 mL of 1 N NaHCO₃ and 20 mL of 1 N hydrochloric acid and then dried over MgSO₄. After evaporation of the solvent, this compound was purified by column chromatography on silica gel using dichloromethane/methanol (95:5) as eluent (0.7 g, 75%). Then, the tert-butyl group of the compound (0.6 g, 1.25 mmol) was removed by the reaction with trifluoroacetic acid (2 mL) in dry dichloromethane for 15 hours. The solvent and excess trifluoroacetic acid was removed under vacuum to get Pyrene-Y acid (0.53 g, 1.25 mmol). Then Pyrene-YL-Otbu was obtained by the peptide coupling reaction with Pyrene-Y acid (0.52 g, 1.22 mmol), L-Leucine tert-butyl ester hydrochloride (0.23 g 1.25 mmol), HBTU (0.47, 1.25 mmol) and 0.58 mL (3.1 mmol) of DIPEA in 10 mL dry DMF. The pure product was obtained (0.38 g, 52%) by column chromatography on silica gel using dichloromethane/methanol (95:5) as eluent. Finally the tert-butyl group of Pyrene-YL-Otbu (0.3 g, 0.506 mmol) was removed by using trifluoroacetic acid to get pure Pyrene-TL (0.27 mg, 0.5 mmol).

¹H NMR (CDCl₃, 400 MHz) δ 0.79-0.83 (m, 6H, CH₃ in leucine moiety), 1.48-1.57 (m, 3H, CH and CH₂ in leucine moiety), 2.7-2.95 (m, 2H, CH₂ in tyrosine moiety), 4.1 (s, 2H, CH₂ at pyrene peptide linker), 4.22-4.28 (m, 1H, chiral CH in leucine moiety), 4.52-4.58 (m, 1H, chiral CH in tyrosine moiety), 6.63 (d, 2H, CH at ortho position of OH group 3J=8.0 Hz), 7.0 (d, 2H, CH at meta position of OH group, ³J=8.0 Hz), 7.86 (d, 1H, pyrene aromatic CH ³J=8.0 Hz), 8.07-8.1 (m, 1H, pyrene aromatic CH), 8.16-8.16 (m, 2H, pyrene aromatic CH and amide NH), 8.18-8.27 (m, 4H, pyrene aromatic CH and amide NH), 8.41-8.43 (m, 3H, pyrene aromatic CH). ESI-MS: m/z: calcd for C₃₃H₃₂N₂O₅: 536.23 [M⁺]; found: 559.27 [M⁺+Na].

Fmoc-FF and Fmoc-FFF was purchased from BiogelX and BACHEM, respectively.

Preparation of Emulsions.

10 mM Fmoc-YL solution was prepared by dissolving 5.32 mg Fmoc-YL in 1 mL phosphate buffer solution. Different volumes of chloroform were added to Fmoc-YL buffer solution at 80° C. (the volume ratio of buffer solution to chloroform is altered from 1:9, 3:7, 5:5, 7:3, to 9:1, total volume was always 1 mL), after hand-shaking for 5 seconds emulsions form in vials. For SEM, IR, stability, average particle size, critical emulsion concentration and demulsification measurements, the volume ratio of buffer solution to chloroform was fixed at 7:3. The concentration of all aromatic peptide amphiphiles studied (Fmoc-YA, Fmoc-YS, Pyr-YL, Fmoc-FF and Fmoc-FFF) was 10 mM, except for in the determination of the critical emulsion concentration (0.1-10 mM) and demulsification measurements (2 mM).

Characterization.

The structures of Fmoc-YL microcapsules were determined by Hitachi S800 field emission scanning electron microscope (SEM) at an accelerating voltage of 10 keV. The transfer of Fmoc-YL, YA and YS were analyzed by UV-Vis spectroscopy (JAS.C.O V-660 spectrophotometer). The formation of Fmoc-YL gels and labeling of ThT were carried out by Fluorescence spectroscopy (JAS.C.O FP-6500 spectrofluorometer). High resolution mass spectra (HRMS) were recorded on a Thermo Electron Exactive. 400.1 (1H) NMR spectra were recorded on Brucker Avance 400 spectrometer at room temperature using perdeuterated solvents as internal standards. The emulsion droplets were characterized by fluorescence microscopy. The devices were mounted on an inverted microscope (AXIO Observer A1, Zeiss) and images were acquired using a EMCCD LucaR camera (Andor Technologies). Images (using brightfield and fluorescence microscopy) were acquired using Zeiss ×20 dry objective and the appropriate filter set for the fluorophore being imaged. Image was analyzed using ImageJ.

The fibrous structures of Fmoc-YL, YA, YS were determined by Atomic Force Microscopy (AFM). The images were obtained by scanning the mica surface in air under ambient conditions using a Veeco diINNOVA Scanning Probe Microscope (VEECO/BRUKER, Santa Barbara, Calif., USA) operated in tapping mode. 20 μl of solutions were placed on a trimmed and freshly cleaved mica sheet (G250-2 Mica sheets 1″×1″×0.006″; Agar Scientific Ltd, Essex, UK) attached to an AFM support stub and left to air-dry overnight in a dust-free environment. The AFM scans were taken at 512×512 pixels resolution. Typical scanning parameters were as follows: tapping frequency 308 kHz, integral and proportional gains 0.3 and 0.5, respectively, set point 0.5-0.8 V and scanning speed 1.0 Hz.

The β sheet-like arrangement was determined by infrared absorption spectra which were recorded on a Bruker Vertex 70 spectrometer, averaging 25 scans per sample at a resolution of 1 cm⁻¹. Samples were sandwiched between two 2 mm CaF₂ windows separated with a 50 μm polytetrafluoroethylene (PTFE) spacer.

Computational Screening:

Peptide structures were obtained via the VMD scripting tool and converted to the MARTINI CG representation using the martinize.py script. 300 molecules of the CG peptide were randomly inserted into a box of dimensions 12.5×12.5×12.5 nm³ and solvated with CG water. 300 ions (Cl⁻ or Na⁺) are added to the system to completely neutralise the charge. The same procedure is carried out for the biphasic system with the addition of 1000 molecules of octane. The addition of the octane was carried out into the water/peptide mixture to give a density approximate to the experimental density of water (999 kg m⁻³). This was achieved by combining a minimised water/tripeptide box with a minimised octane box.

The minimised box was equilibrated for 500,000 steps with a 25 fs time step (12.5 ns simulation time ˜50 ns real time through the scaling of the time due to the softness of the CG potential.⁷ Using Berendsen algorithm to keep temperature (300K) and pressure (1 bar) constant. Periodic boundary conditions are in effect.

Each system was simulated for 9.6 ρs (simulation time) where afterwards the last frame was used to identify the finished structure.

Experimental Validation:

Peptides was purchased at >98% purity form Bachem

Preparation of the peptides were carried out by dissolution of the peptide in water and the pH was altered to a neutral pH ˜7.5.

To each of the systems 100 μL of sudan II labelled rapeseed oil was added. Homogenisation was carried out on each of the sample for 5 secs thereafter; the samples were stored for 24 hrs to ensure a stable emulsion was formed.

FTIR

FTIR samples were contained within a standard IR Harrink Cell between two 2 mm CaF2 windows. A 50 um polytetrafluoroethylene (PTFE) spacer was places between the spacer. Spectra were recorded on a Bruker Vertex70 spectrometer by averaging 25 scans at a spectral resolution of 1 cm⁻¹.

Fluorescence Microscopy

Fluorescence microscope samples were prepared by placing sample on a glass slide with a cover slip placed on top. A drop of silica oil was place on the sample to allow for a lubricated surface. Samples were measured on a Nikon Eclipse E600 upright fluorescent microscope at ×1000 magnification.

Time Stability

These initial observations show samples that showed gelation behaviour, tend to form more stable emulsions that samples that formed other structures. This suggests that the formation of fibrils has a large importance on the peptide ability to self-assemble at the interface forming stable emulsions. The fibrils surround the oil droplets inhibiting coalescence of the droplets. As observed the time stability over a course of 168 hrs showed little or no de-emulsification for KYF, KFF and KYW whereas visible de-emulsification is observed for DFF and FFD peptides, which do not form fibrils. This observation suggests the formation of the fibrils is a major driving force for the stabilisation of emulsions. The importance of the fibrillar formation has shown to be vital for the stabilisation of the emulsions. Comparison of the FTIR before and after emulsification sows the key interactions that help to stabilise the droplets.

The temperature study shows the range of which the emulsion starts to breakdown. It is shown that the KYF is relatively stable through the temperature range. Increase in the temperature between 30-40° C. shows KFF break down. KYW breaks down at slightly higher temperatures approx. 40-50° C. We see that DFF and FFD are relatively de-stabilized at the lower temperatures and the increase in temperature causes minimal change.

Phophatse Experiments

Gel Preparation.

10 mM of synthesised Fmoc-YpL was prepared in 950 μL of 0.6 M sodium phosphate buffer pH 8 and immediately added 50 μL of alkaline phosphatase (0.0555 U·μL⁻¹, or 55.53 U·mL⁻¹ enzymatic concentration), vortexed and subject to ultra sounds at room temperature. All characterisation was done after 24 hours except when stated otherwise. For the Fmoc-YpL precursor, the preparation was the same except no enzyme was added.

Emulsion Preparation.

Fmoc-YpL was prepared in the same way as stated before but in a 5 mM concentration to avoid formation of hydrogels. After 24 hours from the Fmoc-YpL has been prepared in buffer and the alkaline phosphatase added (to assure full dephosphorylation), 500 μL chloroform were added to 500 μL samples and hand-shaken for 5 seconds to make a 50:50 chloroform-in-water emulsion.

On-Demand Activation Test.

To check if the system is switchable on-demand, besides an immediate addition of alkaline phosphatase to the 50:50 chloroform-in water Fmoc-YpL samples, the enzyme was added into the demulsified biphasic system of Fmoc-YpL 1 week, 2 weeks and 1 month after preparation. Photographs were taken and the dephosphorylation assessed by reversed phase HPLC.

Computational

Molecular Dynamic (MD) simulations were carried out in NAMD (NAnoscale Molecular Dynamics) program using the CHARMM force field. Each system was minimised, at 300K, with the steep descent technique and then gradually heated up from 0 to 300 K for 55 ps before being equilibrated for 445 ps, to reach the system stabilization. Finally, the systems were run within an NPT ensemble at 1 atm and 300 K for 200 ns. A 2.0 fs time step was used to integrate Newton's motion equation along with a 12 Å cut-off for the non-bonded interactions. Periodic boundary conditions in the three-dimensional coordinates have been used.

### REFERENCES

(1) Frederix P, W. J. M.; Scott, G. G.; Abul-Haija, Y. M.; Kalafatovic, D.; Pappas, C. G.; Javid, N.; Hunt, N. T.; Ulijn, R. V.; Tuttle, T. Nat Chem 2015, 7, 30.
(2) Frederix, P. W. J. M.; Ulijn, R. V.; Hunt, N. T.; Tuttle, T. The Journal of Physical Chemistry Letters 2011, 2, 2380.
(3) Marrink, S. J.; Risselada, H. J.; Yefimov, S.; Tieleman, D. P.; De Vries, A. H. The Journal of Physical Chemistry B 2007, 111, 7812.
(4) (a) Shimadzu, H.; Suemoto, T.; Suzuki, M.; Shiomitsu, T.; Okamura, N.; Kudo, Y.; Sawada, T. J. Labelled Compd. Radiopharm. 2003, 46, 765; (b) Gebbink, M. F.; Claessen, D.; Bouma, B.; Dijkhuizen, L.; Wösten, H. A. Nature Reviews Microbiology 2005, 3, 333.
(5) (a) Ngai, T.; Behrens, S. H.; Auweter, H. Chem. Commun. 2005, 331; (b) Alava, C.; Saunders, B. R. Colloids and Surfaces A: Physicochemical and Engineering Aspects 2005, 270, 18.
(6) (a) Guzey, D.; McClements, D. J. Adv. Colloid Interface Sci. 2006, 128, 227; (b) F. Gibbs, S. K., Inteaz Alli, Catherine N. Mulligan, Bernard International Journal of Food Sciences and Nutrition 1999, 50, 213.
(7) Marrink, S. J.; de Vries, A. H.; Mark, A. E. The Journal of Physical Chemistry B 2004, 108, 750.

What is claimed is:

1.-33. (canceled)

34. An emulsion comprising a self-assembled network of amphipathic amino acids or peptides formed at an interface between at least two substantially immiscible liquids.

35. An emulsion comprising a self-assembled network of amphipathic peptides according to claim 34 wherein the peptides are between 2-5 amino acids, or 2-4 amino acids in length.

36. An emulsion according to either of claim 34 wherein the peptides are unmodified peptides.

37. An emulsion comprising a self-assembled network of amphipathic amino acids or peptides according to claim 34 wherein the amino acid or the peptide comprise a modified N-terminus.

38. An emulsion comprising a self-assembled network of amphipathic amino acids or peptides according to claim 37 wherein the amino acid or N-terminal amino acid of the peptide comprises a modified group is bound to the amino acid by way of an amide or other suitable bond.

39. An emulsion comprising a self-assembled network of amphipathic amino acids or peptides according to claim 37 wherein the amino acid of peptide is modified to include an aromatic group or groups.

40. An emulsion comprising a self-assembled network of amphipathic amino acids or peptides according to claim 39 wherein the aromatic group or groups comprise a single or multiple ring structures (e.g. polycyclic aromatic hydrocarbons) and/or heterocylic and/or homocyclic ring structures.

41. An emulsion comprising a self-assembled network of amphipathic amino acids or peptides according to claim 40 wherein the aromatic group or groups comprise one, two, three, or four fused ring structures, wherein each ring may be identical or different.

42. An emulsion comprising a self-assembled network of amphipathic amino acids or peptides according to claim 39 wherein said aromatic group or groups comprises anthracene, acenaphthene, fluorene, phenalene, tetracene, pyrene, phenanthrene, naphtalene and chysene, phenylacetyl, as well as heterocyclic structures, such as purine, pyrimidine, pteridine, alloxazine, phenoxazine and phenothiazine.

43. An emulsion comprising a self-assembled network of amphipathic amino acids or peptides according to claim 41 wherein the aromatic group or groups are bonded to said amino acid through a substituent present on one or more of the aromatic rings.

44. An emulsion comprising a self-assembled network of amphipathic amino acids or peptides according to claim 43 wherein the substituent comprises a reactive C1-C4 alkyl, alkyloxy, alkylamino, phosphate, carboxylic acid, amino, alcohol, N-hydroxysuccinimide, hydroxybenzotriazole, halide, or 1-Hydroxy-7-azabenzotriazole.

45. An emulsion comprising a self-assembled network of amphipathic amino acids or peptides according to claim 40 wherein the aromatic group or groups comprise furene, pyrene, purine and pyrimidine containing structures, such as fluorenylmethyloxycarbonyl (FMOC), 9-fluorenylmethyl succinimidyl carbonate (FMOC-)Su), C1-C4 alkyl substituted pyrene, and natural and synthetic nucleotides

46. An emulsion comprising a self-assembled network of amphipathic amino acids or peptides according to claim 34 wherein the amino acid or peptide comprise a modification at the N-terminus, C-terminus and/or on the peptide backbone, such as where one or more amino acids are phosphorylated.

47. An emulsion comprising a self-assembled network of amphipathic peptides according to claim 34 comprising or consisting of the sequence YL, YA, YS, FF, FFF and YL (using the common one-letter code to identify the amino acids).

48. An emulsion comprising a self-assembled network of amphipathic peptides according to claim 46 which have been modified at the N-terminal amino acid (i.e. Y in the case of YL, for example) to include an FMOC or pyrene group.

49. An emulsion comprising a self-assembled network of amphipathic peptides according to claim 34 comprising or consisting of the sequence KYW, KFF, KYF, FFD, or DFF.

50. An emulsion comprising a self-assembled network of amphipathic amino acids or peptides according to claim 34 wherein the emulsion remains stable over a period of at least 1 week, 2 weeks, 4 weeks, 2 months, 6 months, 12 months or more when not in the presence of salt.

51. A method of making an emulsion, the method comprising mixing at least 2 substantially immiscible liquids in the presence of an amino acids or peptides as described in claim 34, in order to form an emulsion.

52. The method of making an emulsion according to claim 51 comprising mixing at least 2 substantially immiscible liquids in the presence of a phosphorylated peptide and a phosphatase, in order to form an emulsion.

53. The emulsion according to claim 34 wherein the emulsion further comprises an agent, selected from the group consisting of a pharmaceutical agent, dye, flavour enhancer and a pesticide.

54. A food product comprising an emulsion according to claim 34, wherein the emulsion comprises a food grade oil.

55. The food product according to claim 54 wherein the emulsion comprises a food grade oil, selected from the group consisting of an edible oil or fat, in particular a vegetable oil or fat, such as coconut oil, palm oil, palm kernel oil, olive oil, soybean oil, canola oil (rapeseed oil), pumpkin seed oil, corn oil, sunflower oil, safflower oil, peanut oil, grape seed oil, sesame oil, argan oil, rice bran oil and other vegetable oils, as well as animal-based oils including butter and lard.

56. A method of virtually identifying a peptide as being capable of emulsion formation, the method comprising selecting a propensity of aggregation (AP*) at an organic solution/aqueous solution interface, the method comprising initially selecting a peptide which displays a propensity of aggregation (AP) in aqueous solution and thereafter selecting a peptide for its propensity of aggregation (AP*) at an organic solution/aqueous solution interface.

57. The method according to claim 56 wherein in addition or alternatively to AP determination, a hydrophilicity-adjusted measure of propensity for aggregation (AP_{H}) for the peptide is determined, the AP_{H} being determined by adjusting a measure of propensity of aggregation (AP) for the peptide in dependence on a measure of hydrophilicity for the peptide.

58. The method according to claim 57 wherein determining AP_{H} for the peptide comprises using the equation: AP_{H}=(AP′)^{α}(log P)′ wherein α is a numerical constant, having a value between 0.5 and 5, optionally between 1 and 4, further optionally between 1 and 3, log P is the measure of hydrophilicity for the peptide, and an apostrophe denotes normalisation.

59. The method according to claim 56 further comprising synthesising peptides which display a AP* at an organic solution/aqueous solution interface and optionally testing such synthesised peptides for their ability to aggregate an organic solution/aqueous solution interface, or capability of forming an emulsion between an organic and an aqueous solution.

60. A method for virtually screening peptides (for example, tripeptides) comprising screening large numbers of peptides by calculating AP/AP_{H} for each peptide, and identifying a plurality of peptides based on the calculated AP/AP_{H}.

61. The method according to claim 60 wherein peptides identified as having a high and/or above threshold AP/AP_{H} value are selected for AP* determination.

62. The method according to claim 61 further comprising displaying the AP* for the peptide from simulation.

63. The method according to claim 61 wherein the AP* for each of the plurality of peptides is determined from a respective simulation, which may comprise a molecular dynamics simulation.

64. The method according to claim 63 wherein the AP/AP_{H}/AP* for a given peptide comprises a ratio between a solvent accessible surface area at the beginning of the molecular dynamics simulation and a solvent accessible surface area at the end of the molecular dynamics simulation.

65. A method of producing a peptide aggregate comprising a peptide capable of self-aggregation at an organic solution/aqueous solution interface, the method comprising identifying a peptide by determining a measure of propensity of aggregation (AP) for the peptide in aqueous solution, optionally in dependence on a measure of hydrophilicity (AP_{H}) for the peptide, identifying by simulation whether or not the peptide is capable of self-aggregation at an organic solution/aqueous solution interface, and synthesising a peptide identified as being capable of self-aggregation at an organic solution/aqueous solution interface.
